(19) United States
(12) Patent Application Publication
(10) Pub. No.: US 2009/0221530 A1
(43) Pub. Date: Sep. 3, 2009

(54) Relaxase Modulators and Methods of Using Same

(76) Inventors: Matthew R. Redinbo, Chapel Hill, NC (US); Scott Lujan, Chapel Hill, NC (US); Steven W. Matson, Chapel Hill, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/084,843

(22) PCT Filed: Nov. 8, 2006
(86) PCT No.: PCT/US06/43703

§ 371 Date: Feb. 17, 2009

Related U.S. Application Data
(60) Provisional application No. 60/734,878, filed on Nov. 9, 2005.
(60) Provisional application No. 60/787,860, filed on Mar. 31, 2006.

(51) Int. Cl. A61K 31/665; C12Q 1/68

(52) U.S. Cl. 514/99; 435/6

(57) ABSTRACT

Methods of treating a microbial infection in a subject by administering to the subject an effective amount of a compound that modulates an enzymatic activity of a relaxase polypeptide is provided. Methods of inhibiting bacterial conjugation by modulating activity of a relaxase polypeptide in a bacterium are also provided. Novel compounds that modulate relaxase enzymes and assays for measuring kinetics of relaxase enzymes and selecting for modulators of relaxase enzyme activity are further provided.

### RELATED APPLICATIONS

This application claims the benefit of U.S. Patent Application Ser. No. 60/787,860, filed Mar. 31, 2006, and 60/734,878, filed Nov. 9, 2005, the disclosures of each of which are incorporated herein by reference in their entireties.

### GOVERNMENT INTEREST

This presently disclosed subject matter was made with U.S. Government support under Grant No. R01 CA90604 awarded by the National Institutes of Health. The U.S. Government has certain rights in the subject matter disclosed herein.

### TECHNICAL FIELD

The presently disclosed subject matter relates to compounds and methods for modulating relaxase enzyme activity. More specifically, the presently disclosed subject matter relates to relaxase inhibitor compounds and methods for the treatment of microbial infections and inhibiting bacterial conjugation, as well as assay methods related to the same.

### BACKGROUND

Nearly sixty years ago, Lederberg and Tatum first established that genetic elements could move directly between microbial cells, and in the process discovered both the E. coli F plasmid and founded bacterial genetics (Lederberg and Tatum, 1946). It is now known that this process, DNA conjugation, requires close cell-to-cell contact and mediates the majority of the horizontal transfer of genes between bacteria (Firth et al., 1996; Lanka and Wilkins, 1995; Llosa et al., 2002; Pansegrau and Lanka, 1996; Sprague, 1991; Zechner et al., 2000).

Conjugative transfer is the main route by which antibiotic resistance genes or virulence factors are propagated in bacteria, which leads to the development of multi-drug resistant variants (Ahmed et al., 2005; Wei et al., 2005) and to the pathogenization of previously innocuous strains (Golubov et al., 2004; Oancea et al., 2004). In clinical settings, it has been shown that antibiotic resistance can be rapidly acquired in epidemic bacterial infections and that this acquisition is dependent on DNA conjugation (Ahmed et al., 2005; Domart et al., 1974; Tosini et al., 1998; Wei et al., 2005). Currently, there are no known methods for inhibiting DNA conjugation.

Thus, inhibitors of DNA conjugation are urgently needed to prevent the further spread of antibiotic resistance genes and virulence factors between bacteria, especially as the stock of useful antibiotics continues to dwindle worldwide. Further, new novel antimicrobial agents are desperately needed to replace therapeutics no longer effective due to conjugation-mediated antibiotic resistance.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In one embodiment of the presently disclosed subject matter, a method of treating a microbial infection in a subject is provided. The method comprises administering to the subject an effective amount of a compound that modulates an enzymatic activity of a relaxase polypeptide. In some embodiments, the microbial infection treated is a bacterial infection and the relaxase polypeptide is a Mob polypeptide, such as for example a TraI polypeptide. In embodiments wherein the method is treating a bacterial infection, the compound can inhibit polynucleotide cleavage, polynucleotide religation, or both polynucleotide cleavage and polynucleotide religation enzymatic activities of the relaxase polypeptide. In some embodiments, the microbial infection treated is a viral infection and the relaxase polypeptide is a Rep polypeptide. In embodiments where the microbial infection treated is a viral infection, the compound can inhibit replication of viral polynucleotide sequences. In some embodiments, the compound is co-administered with at least one additional compound having antimicrobial activity. In some embodiments, the subject is a mammal, including for example humans.

In another embodiment of the presently disclosed subject matter, a method of inhibiting bacterial conjugation is provided. The method comprises contacting a relaxase polypeptide within a bacterium with a relaxase dependent antibiotic, wherein the antibiotic modulates an enzymatic activity of the relaxase polypeptide. In some embodiments, the relaxase polypeptide is a Mob polypeptide, such as for example a TraI polypeptide. In some embodiments, the antibiotic inhibits polynucleotide cleavage, polynucleotide religation, or both polynucleotide cleavage and polynucleotide religation enzymatic activities by the relaxase polypeptide. In some embodiments, the antibiotic is co-administered to the bacterium with at least one additional antibiotic.

The presently disclosed subject matter further provides compounds, including novel compounds, for use with the methods disclosed herein. In some embodiments, the compounds disclosed herein have a net negative charge, and in some embodiments, the compounds have a −2 charge. In some embodiments the compound comprises a phosphate, carboxylate, sulfate, or nitro moiety, which can in some embodiments be a bis-moiety (e.g., a bis-phosphate moiety).

In some embodiments, the relaxase modulating compound has the structure of Formula (I):

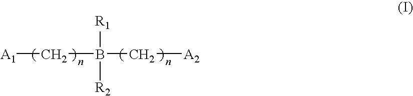

wherein:

n is an integer from 0 to 4;

A₁ and A₂ are independently selected from the group consisting of H, hydroxyl, alkyl, substituted alkyl, cycloalkyl, substituted cycloalkyl, aryl, substituted aryl, aralkyl, substituted aralkyl, phosphate, carboxylate, sulfate, and nitro, provided that at least one of A₁ or A₂ is phosphate, carboxylate, sulfate, or nitro;

B is selected from the group consisting of N, alkylene, substituted alkylene, cycloalkylene, substituted cycloalkylene, cycloalkenylene, substituted cycloalkenylene, arylene, and substituted arylene; and

R₁ and R₂ can each be present or absent and are independently selected from the group consisting of H, hydroxyl, halo, alkyl, substituted alkyl, cycloalkyl, substituted cycloalkyl, aryl, substituted aryl, aralkyl, and substituted aralkyl;

or a pharmaceutically acceptable salt thereof.

In some embodiments, the relaxase modulating compound of Formula I is selected from the group consisting of imidodiphosphate, methylenediphosphonate, etidronate, clodronate, pamidronate, alendronate, neridronate, iminobis, N-(2-hydroxyethyl)iminobis, glyphosine, 1,2-bis(dimethoxyphosphoryl)benzene, dichloromethylenediphosphonate, and SR12813 (3,5-di-tert-butyl-4-hydroxystyrene-β,β-diphosphonic acid tetraethyl ester). Further, in some embodiments, the relaxase modulating compound of Formula I has a structure selected from the group consisting of:

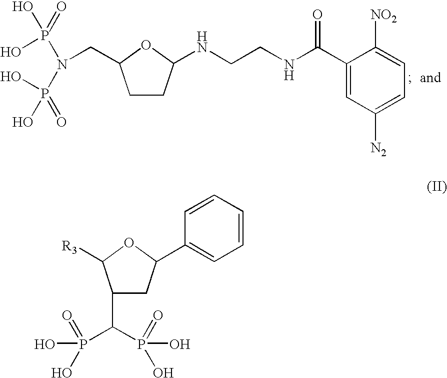

wherein:

R₃ is selected from the group consisting of H, hydroxyl, alkyl, substituted alkyl, cycloalkyl, substituted cycloalkyl, aryl, substituted aryl, aralkyl, and substituted aralkyl.

In another embodiment, the presently disclosed subject matter provides an assay method for measuring multiple catalytic kinetic time courses of a multifunctional polynucleotide-specific enzyme. In some embodiments the method comprises (a) providing a multifunctional polynucleotide-specific enzyme; a first substrate polynucleotide comprising a capture tag linked to a first end of the first polynucleotide, an enzyme recognition polynucleotide sequence, and a label linked to a second end of the first polynucleotide; and a second substrate polynucleotide comprising an enzyme recognition polynucleotide sequence and a cleavable capture tag linked to an end of the second polynucleotide; (b) incubating the enzyme with the first polynucleotide and the second polynucleotide for a time sufficient to permit the enzyme to react with the first polynucleotide and the second polynucleotide; (c) capturing the first polynucleotide and the second polynucleotide to a capture affinity molecule having binding affinity for both the capture tag and the cleavable capture tag, wherein the capture affinity molecule is bound to a substrate; (d) washing the substrate to remove uncaptured molecules; (e) determining a first kinetic time course of the enzyme based on a measured change in an amount of the label bound to the substrate over a time course; (f) cleaving the cleavable capture tag, thereby releasing the second polynucleotide from the substrate; and (g) determining a second kinetic time course of the enzyme based on a measured change in an amount of the label bound to the substrate before and after cleavage of the cleavable capture tag over a time course.

In some embodiments, the multifunctional polynucleotide-specific enzyme is a relaxase enzyme, such as for example a Mob relaxase enzyme. In some embodiments the capture tag is biotin and the cleavable capture tag is photocleavable biotin. In these embodiments in particular, the capture affinity molecule is streptavidin. In some embodiments, the label is a fluorescent label and the label can be bound in some embodiments to an end of a probe oligonucleotide having sequence homology to the second end of the first polynucleotide, wherein the probe oligonucleotide can hybridize to the first polynucleotide and thereby link the label to the first polynucleotide. In some embodiments, the enzyme recognition polynucleotide sequence comprises a polynucleotide sequence homologous to a bacterial oriT polynucleotide sequence, and in some embodiments the enzyme recognition polynucleotide sequence is a bacterial oriT polynucleotide sequence. In some embodiments of the assay method, the enzyme reacts with the first and second polynucleotides to cleave the polynucleotides, crossover ligate the polynucleotides, or both. In some embodiments, determining the first and second kinetic time courses of the enzyme comprises determining the Vₘₐₓ, Kₘ, or both of the enzyme reactions with the first and second polynucleotides. Further, in some embodiments, the first kinetic time course is a measure of cleavage of the first polynucleotide by the enzyme and the second kinetic time course is a measure of crossover ligation of the first polynucleotide and the second polynucleotide by the enzyme.

In yet another embodiment of the presently disclosed subject matter, a method of selecting for inhibitors of polynucleotide-specific enzymes is provided. In some embodiments the method comprises (a) contacting a polynucleotide-specific enzyme with a substrate polynucleotide comprising a label in the presence of a candidate inhibitor; (b) incubating the enzyme and the polynucleotide in the presence of the candidate inhibitor for a time sufficient to permit the enzyme to catalytically react with the polynucleotide; (c) measuring a change in an amount of the labeled polynucleotide present over time, whereby the change in the amount of labeled polynucleotide correlates with an activity of the enzyme on the polynucleotide; and (d) selecting the candidate inhibitor as an inhibitor of the enzyme if the activity of the enzyme on the polynucleotide is reduced in the presence of the candidate inhibitor as compared to a reaction in which the candidate inhibitor is absent. In some embodiments, the polynucleotide-specific enzyme is a relaxase enzyme, such as for example a Mob relaxase enzyme. In some embodiments, the enzyme recognition polynucleotide sequence comprises a polynucleotide sequence homologous to a bacterial oriT polynucleotide sequence, and in some embodiments the enzyme recognition polynucleotide sequence is a bacterial oriT polynucleotide sequence. In some embodiments, the label is a fluorescent label and in some embodiments the label is bound to an end of a probe oligonucleotide having sequence homology to the polynucleotide, wherein the probe oligonucleotide can hybridize to the polynucleotide and thereby link the label to the polynucleotide. In some embodiments, the enzyme reacts with the polynucleotide to cleave the polynucleotide, ligate the polynucleotide, or both. In some embodiments, the method further comprises determining the Kᵢ, the mechanism of inhibition, or both, of the inhibitor on the enzyme.

Accordingly, it is an object of the presently disclosed subject matter to provide for the modulation of relaxase enzyme activity. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter and non-limiting examples.

### BRIEF DESCRIPTION OF THE DRAWINGS

FIG. 1 is a schematic core representation of the Pilot and Pump relaxases, which exhibit a similar overall fold but distinct structural and functional features. The central β-sheet is common to all Mol (mobilization) and Rep (replication) relaxases, while transparent elements are less stringently conserved. Pilots (left) contain a single catalytic tyrosine (Y) and remain covalently attached via a 5′-phosphotyrosine linkage to the ssDNA transferred into the recipient cell during conjugation. Pilot relaxases are sometimes fused to secondary catalytic domains, such as primase fusions in some IncQ relaxases. The IncQ, P, I, X and Ti tra and vir conjugative plasmids, for example, utilize pilot-type relaxases. Pumps (right), in contrast, contain up to five tyrosines (most commonly four, Y₁-Y₄) and are fused to highly efficient helicase domains. These enzymes remain within the donor cell and transfer ssDNA by pumping it through the conjugative septum. The IncW, N, P9 and F conjugative plasmids employ pump-type relaxases (the IncFI F plasmid relaxase TraI is disclosed herein). Also noted on the pump schematic are a conserved aspartic acid (TraI D81) and tryptophan (TraI W277). Both pilot and pump relaxases contain a conserved three-histidine motif that coordinates an ion containing a 2⁺ charge.

FIGS. 2A-2C schematically depict the molecular structure of the relaxase TraI.

FIG. 2A schematically depicts results of a crystal structure determination of the N300 region of the F plasmid relaxase TraI. α-helices A-J and β-strands 1-11 are labeled, as is the single thymine nucleotide from the −1 position of the oriT sequence visualized in the structure. Residues 66-72 and 236-266 (with the exception of 264-265) are disordered, although the active site and its four adjacent tyrosine residues are observed (with Y16 mutated to phenylalanine in this complex: Y16F, Y17, Y23, Y24). Helices A and A′ are in ribbon format.

FIG. 2B depicts a stereoview schematic representation of the active site of the TraI relaxase disclosed herein (see Table I). The four tyrosines (Y16F, Y17, Y23, Y24) and a conserved aspartic acid (D81) are in close proximity to the Mg ion bound by three histidine side chains (H146, H157, H159). Simulated-annealing omit F_{obs}-F_{calc} electron density −1 thymidine is contoured at 2.5 σ (green).

FIG. 2C depicts a stereoview schematic representation of the superposition of the active site of the TraI relaxase structure disclosed herein (orange) on that of the TrwC relaxase structure (blue; PDB 1OMH). Only two of the four tyrosine side chains of the TrwC structure are observed in this complex, and those visible occupy distinct positions relative to TraI; for example, the first tyrosine of TrwC (Y18) superimposes on the second tyrosine of TraI (Y17), while the second TrwC tyrosine (Y19) is in a unique position. Note the similar positions of the TraI and TrwC thymidine 3′-hydroxyl.

FIG. 3 shows a comprehensive mechanism model for F plasmid DNA transfer catalyzed by the pump-type conjugative relaxase TraI. TraI, which contains both a relaxase and helicase region, binds to and unwinds the F plasmid oriT when the relaxosome is present (Steps 1-2). The first catalytic tyrosine (1Y; Y16 in TraI) nicks the ssDNA and leaves a free 3′-hydroxyl (Step 3), which serves as the substrate for generating conjugation-lined replacement DNA strand by a replisome at the oriT (Step 4). DNA unwinding by the helicase region of the relaxase extrudes the T-strand of the F plasmid into the recipient cell while the 5′-end is still covalently attached to the Y16 via a 5′-phosphotyrosine intermediate (Step 4). After the hybrid oriT formed by the replisome returns to the relaxase (Step 5), the second catalytic tyrosine (3Y; Y23 in TraI) nicks the new DNA strand to generate a 3′-hydroxyl that then reverses the Y16-mediated phosphotyrosine intermediate (Step 6). The 3′-hydroxyl generated by trailing replication reverses the second phosphotyrosine intermediate on Y-23 (Step 7), releasing the intact T-strand into the recipient cell and leaving a dsDNA F plasmid (Step 8). If strand replacement is not initiated at the oriT, the relaxase would return to an oriT half-site that leads to the direct reversal of the first 5′-phosphotyrosine intermediate at Y16 (Step 5′). In this case, replacement synthesis (labeled in pink) is initiated at the F plasmid oriV origin of vegetative replication. Note that the conjugation-linked strand synthesis pathway (Steps 4-8; green arrows) requires two catalytic tyrosines (Y16, Y23); in contrast, when conjugation is not linked to strand replacement (Step 4-5′; pink arrows) only one catalytic tyrosine is necessary.

FIGS. 4A-4D shows data resulting from examination of the kinetics of DNA cleavage and crossover catalyzed by TraI.

FIG. 4A shows a schematic representation of the fluorescence-based assay employed to examine the kinetics of DNA cleavage and crossover mediated by TraI N300. In the Examples, oligonucleotide a is designated ‘PCb31eco’, oligo b is ‘b29’, and oligo c is ‘downF’.

FIG. 4B is a graph showing kinetics of DNA cleavage by the N300 region of TraI measured alone and in the presence of two concentrations of imidodiphosphate (also referred to as imidobisphosphate or pNp; 1 and 10 nM). The chemical structure of pNp is shown in the inset of FIG. 4B.

FIG. 4C depicts a stereoview schematic representation of the 3.0 Å resolution crystal structure the TraI N300-DNA nucleotide-pNp complex with the observed pNp phosphate group shown. The green mesh is the simulated annealing omit F_{obs}-F_{calc} electron density peak at 3.0 σ into which one pNp phosphonate moiety was modeled.

FIG. 4D is a graph showing imidodiphosphate (pNp) impacts cell viability and DNA conjugation in vivo in a TraI-dependent manner. Cells that lack the F plasmid (JS4, F−) are resistant to pNp (maroon), while the same cells including the F plasmid (JS10, F+) are effectively killed by the compound (yellow). However, if TraI is removed from F+ cells (JS11, F+/ΔtraI), they become resistant to pNp (orange). Finally, DNA conjugation from F+ (JS10) cells to F− (JS4) cells is also effectively inhibited by pNp (green), with the compound exhibiting an EC50 value of 10 μM.

FIGS. 5A-5C shows schematic representations of catalytic and non-catalytic tyrosine positioning in pump-type relaxases.

FIG. 5A depicts a stereoview schematic representation of the active site of the TraI relaxase low salt structure presented here (with the path of the ssDNA observed in the TrwC structure 1 OMH shown as a cyan tube), flanked by close-up views of the Y16/17 and Y23/24 tyrosines on the right and left, respectively.

FIG. 5B depicts a stereoview schematic representation of the TrwC relaxase active site (with two observed positions of Thy25, the −1 thymine nucleotide, shown for structures 1OMH and 1 QX0), flanked by the close-ups of the first two tyrosines (Y17/18) and the second two tyrosines observed in these structures.

FIG. 5C depicts a stereoview schematic representation of the TraI relaxase active site containing two 5′-phosphotyrosine intermediates and a ssDNA ending in a free 3′-hydroxyl, flanked by close-up views of the TraI tyrosines. Y16/17 are positioned similar to TrwC Y17/18 in B; Y23/24 are positioned as they would appear if αA were extended by one helical turn relative to A. For all panels, the bound divalent metal ion is rendered as a blue sphere, locations of expected phosphates and purple circles, and the limits of a putative path for extended DNA strands are indicated by two cyan arrows.

FIG. 6 shows the chemical structures of several exemplary relaxase modulating compounds of the present disclosure.

FIG. 7 illustrates a “mix-and-measure” in vivo assay based on oxygen quenching of a gel-embedded fluorophore for measurement of cell survival and conjugative DNA transfer.

### BRIEF DESCRIPTION OF THE TABLES

Table I shows crystallographic statistics for N300 structures. Values for the highest resolution shell are in parentheses. R_{sym}=Σ|I− [symbol] I [symbol] |/ΣI, where/is the observed intensity and [symbol] I [symbol] is the average intensity of multiple symmetry-related observations of that reflection. R_{factor}=Σ∥Fₒ|−|F_{c}∥/Σ|Fₒ|, where Fₒ and F_{c} are the observed and calculated structure factors, respectively. R_{free}=Σ∥Fₒ|−|F_{c}∥/Σ|Fₒ|, for 7% of the data which was not used in structural refinement.

Table II shows activities of F plasmid TraI relaxase mutants relative to wild type. n.d.=value not determined.

Table III shows kinetic constants for oriT cleavage and crossover by F plasmid TraI N300. Vₘₐₓ is the apparent maximum reaction velocity and Kₘ is the apparent Michaelis constant for each reaction. K_{ic} and Kᵢᵤ are competitive and uncompetitive inhibition constants, respectively. Values used to calculate the reported constants were derived from either nonlinear regression with the Michaelis/Menton equation (M/M NLR) or by the Cornish-Bowden/Eisenthal direct linear plot method (C-B/E DLP). a=value not reported due to poor regression statistics.

Table IV shows the phylogenetic relationship between Pilot and Pump relaxases. The point at which helicase fusion, tyrosine bifurcation (duplication) and a large domain insertion occurred are indicated. The E. coli F plasmid TraI enzyme is a member of the IncF family, which is listed at the bottom of Table IV. Residues or residue pairs conserved in at least 80% of taxa within a cluster are listed in capitals, with the catalytic tyrosines underlined in bold. Key residues are also indicated, with ‘-’ for alignment gaps, ‘c’ for charged, ‘p’ for polar, or ‘s’ for small (serine, alanine, glycine, threonine, cysteine, or valine). If no conservation is observed, the residue is encoded as an ‘x’. Residues 33, 52 and 144 (*) form the pocket in which Tyr-17 is proposed to dock during catalysis, while residue 277 (**) is proposed to contact Tyr-24.

Table V shows the protein sequences used to generate the cladogram shown in Table IV, along with their NCBI accession codes.

Table VI shows EC₅₀ and MIC data from a fluorescence assay for measuring antimicrobial activity of relaxase inhibitor compounds.

### BRIEF DESCRIPTION OF THE SEQUENCE LISTING

SEQ ID NO: 1 is a 9-base single-stranded DNA oligonucleotide derived from the F plasmid oriT, which can be recognized and cleaved by a relaxase enzyme.

SEQ ID NO: 2 is an oligonucleotide utilized for the kinetic assays disclosed herein, which can be 5′ end labeled with a biotin molecule.

SEQ ID NO: 3 is an oligonucleotide utilized for the kinetic assays disclosed herein, which can be 5′ end labeled with a photocleavable biotin molecule.

SEQ ID NO: 4 is a fluorescently labeled oligonucleotide probe utilized for the kinetic assays disclosed herein.

### DETAILED DESCRIPTION

Conjugation, the direct transfer of genetic material between cells, is the central route by which antibiotic resistance genes and other virulence factors are propagated in bacteria. The acquisition of multidrug resistance occurs quickly in epidemic bacterial infections; however, no methods currently exist to combat such propagation in vitro or in vivo.

Conjugative relaxases initiate the transfer of DNA from donor to recipient cells by nicking DNA strands and forming phosphotyrosine intermediates. The examination of DNA cleavage, religation and transfer actions of the exemplary conjugative E. coli F plasmid relaxase TraI by x-ray crystallography, site-directed mutagenesis, and novel kinetic assays is disclosed herein. Using these data, the first comprehensive and broadly applicable mechanism for conjugative DNA transfer has been developed and is disclosed herein. This mechanism explains, for the first time the unique features of this class of conjugative relaxases, including their fused helicases and clusters of multiple tyrosines.

In addition, based on unique predictions formulated from this proposed mechanism, the first class of inhibitors of conjugative relaxases is identified herein and shown to impact both DNA cleavage and religation in vitro with nanomolar affinity, and to inhibit conjugation and provide antimicrobial activity in vivo. Although the model E. coli F plasmid system utilized herein does not transfer any antibiotic resistance genes, plasmid systems that do mediate the propagation of antibiotic resistance (e.g., R1, R100, R388, R46; Table IV and V) contain pump-type relaxases up to 98% identical to F plasmid TraI.

Thus, the relaxase inhibitor compounds disclosed herein provide the first interventions for the development of antibiotic resistance in clinical settings. In addition, because for example the tyrosine- and metal ion-dependent portion of the mechanism of ssDNA strand cleavage appears conserved between all members of the Mob conjugative relaxase family and the Rep viral relaxase family, such compounds also have broad and potent antibiotic and antiviral activities and therefore are useful in methods of treating both bacterial and viral infections in subjects.

### I. GENERAL CONSIDERATIONS

Relaxases are key enzymes in conjugative transfer and are required for the mobilization of DNA plasmids, transposons, insertion elements, and other genetic packages (Byrd and Matson, 1997; Lanka and Wilkins, 1995; Pansegrau and Lanka, 1996). At a minimum, these enzymes are responsible for nicking one strand of the transferred (T) DNA at the initiation of the mobilization process (Byrd and Matson, 1997), the first step in conjugation, and may play further roles in both mobilization and transfer (Llosa et al., 2002: Matson et al., 2001; Matson and Ragonese, 2005). Conjugative DNA transfer requires two large macromolecular complexes—the relaxosome and a type IV secretion system (T4SS). The F plasmid relaxosome is composed of three plasmid-encoded proteins, TraI, TraY and TraM, and one host-encoded protein, the integration host factor (IHF). TraI contains both a relaxase and a helicase region located within the N- and C-terminal regions, respectively, of this 1,756 residue protein. F plasmid DNA is nicked by the relaxase region of TraI at the plasmid's origin of transfer (oriT), and the nicked single strand is transferred to the recipient cell through the T4SS-mediated conjugative septum. The energy required to drive DNA transfer can be provided by the helicase region of TraI. Although transfer is remarkably efficient, requiring only approximately five minutes to complete, it is not clear at what stage the cellular replication machinery initiates replacement strand synthesis and converts the ssDNA in both the donor and recipient cell to dsDNA.

Members of the Mob (mobilization) family of conjugative relaxases utilize active site tyrosine residues to catalyze metal-ion dependent transesterification reactions generating long-lived covalent 5′-phosphotyrosine intermediates and free 3′-hydroxyls (Byrd and Matson, 1997; Lanka and Wilkins, 1995; Pansegrau and Lanka, 1996). The determination of the first Mob relaxase crystal structures (Datta et al., 2003; Guasch et al., 2003) confirmed previous hypotheses that they are structurally related to the Rep (replication) family relaxases involved in viral and plasmid rolling-circle-replication (RCR) (Campos-Olivas et al., 2002; Dyda and Hickman, 2003; Hickman et al., 2002; Ilyina and Koonin, 1992; Waters and Guiney, 1993). Thus, Mob and Rep relaxases share several functional traits, including metal-dependent site- and strand-specific DNA transesterification activities involving tyrosine nucleophiles.

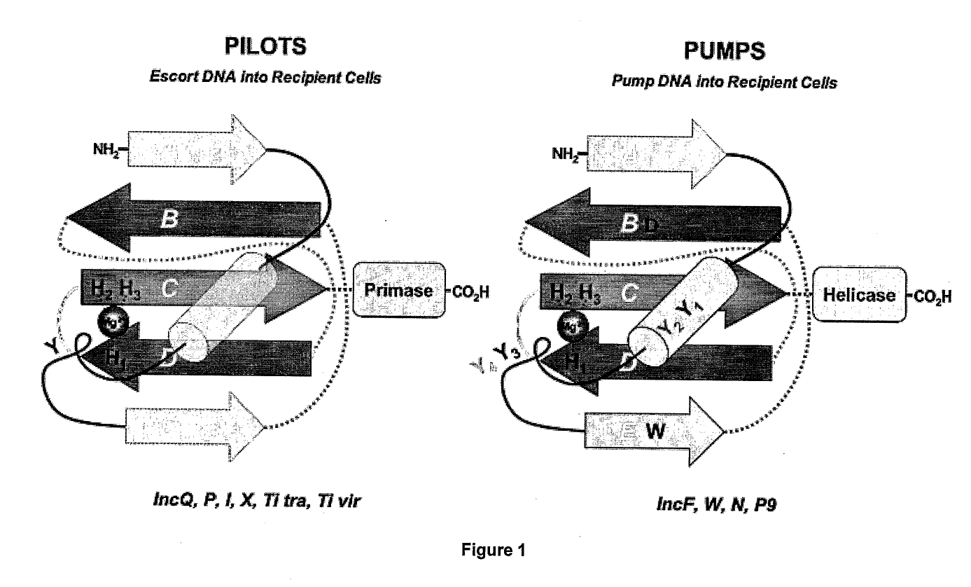

The Mob family of conjugative relaxases can be divided into two distinct classes based on sequence, structure and functional data. These classes are described herein as “pilots” (as proposed earlier by Llosa et al., 2002) and “pumps” (FIG. 1). The relaxases predominantly encoded on the conjugative plasmids of incompatibility groups IncQ, P, I, X, and on plasmid pTi/Ri/AT ‘tra’ and ‘vir’ operons, are thought to accompany or “pilot” the covalently bound ssDNA into the recipient cell, as demonstrated experimentally for the pTi relaxase VirD2 (Gelvin, 2000). These enzymes contain only a single catalytic tyrosine. In contrast, the “pump” relaxases, which include enzymes encoded on the conjugative plasmids of IncF, W, N and P9 incompatibility classes, remain in the donor cell and pump the transferred ssDNA into the recipient cell across a conjugative septum. Indeed, no intercellular protein transport has been observed in IncF systems under conditions where such transfer was noted for a pilot relaxase (Rees and Wilkins, 1990). Pump relaxases always contain at least a second conserved tyrosine within the active site (and frequently up to four or five total tyrosines), and are fused to a highly processive C-terminal helicase domain that provides the motive force for DNA transfer (Abdel-Monem et al., 1976; Dash et al., 1992; Lahue and Matson, 1988; Matson et al., 2001). Two of the active site tyrosines have been shown to be catalytic and to form transient covalent bonds with the DNA during strand cleavage, religation and transfer (Grandoso et al., 2000). Thus, pump-type relaxases are more complex, multifunctional enzymes relative to the simpler pilot-type proteins.

To date, no comprehensive mechanism for the concerted catalytic and non-catalytic steps of relaxase function has been presented that accounts for the available structural and functional data. As disclosed in detail herein, a multidisciplinary approach has been taken to provide a complete picture and assemble a catalytic and DNA transfer mechanism for the pump-type conjugative relaxases. In particular, the presently disclosed subject matter addresses the roles played by the multiple (up to five) tyrosines maintained in the active sites of these enzymes. Although it has been shown that two of these tyrosines are catalytic, it has heretofore not been clear why the remaining side chains are conserved as tyrosines, and whether both catalytic residues are always required. Further, the presently disclosed subject matter provides data as to the importance of the 2+ charge on the bound metal ion and its role in catalysis.

Using structural, functional, mutagenesis and kinetic data applied to the F plasmid TraI relaxase as a model system, a comprehensive DNA transfer mechanism is provided and, relaxase inhibitors are described and shown to act in vitro and in vivo. Taken together, these observations lead to the first strategies for limiting the propagation of antibiotic resistance in clinical settings and for the treatment of microbial infections.

### II. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Throughout the specification and claims, a given chemical formula or name shall encompass all optical and stereoisomers, as well as racemic mixtures where such isomers and mixtures exist.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Further, as used herein, the terms “alkyl” and/or “substituted alkyl” include an “allyl” or an “allylic group.” The terms “allylic group” or “allyl” refer to the group —CH₂HC═CH₂ and derivatives thereof formed by substitution. Thus, the terms alkyl and/or substituted alkyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.”

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise for example phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

A structure represented generally by a formula such as:

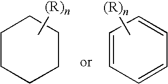

as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

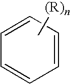

wherein n is an integer from 0 to 2 comprises compound groups including, but not limited to:

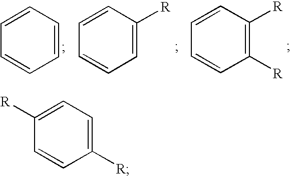

and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons in some embodiments.

“Arylene” refers to a bivalent aryl group, as “aryl” is defined herein. An exemplary arylene would be phenylene, which can have ring carbon atoms available for bonding in ortho, meta, or para positions with regard to each other, i.e.,

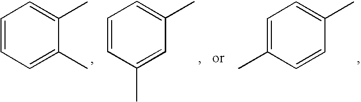

respectively. The arylene group can be optionally substituted (a “substituted arylene”) with one or more “aryl group substituents” as defined herein, which can be the same or different.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined herein, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

“Cycloalkylene” refers to a bivalent cycloalkyl, as “cycloalkyl” is defined herein. The cycloalkylene group also can be optionally substituted (a “substituted cycloalkylene”) with an “alkyl group substituent”, as defined herein.

The term “cycloalkenylene” refers to a divalent unsaturated or partially unsaturated cyclic hydrocarbon, including, but not limited to, a C3-C20 cyclic hydrocarbon, having one or more carbon-carbon double bonds, provided that the one or more carbon-carbon double bonds do not form an aromatic ring system. Representative cycloalkenylene groups include, but are not limited to, cyclopentenylene, cyclohexenylene, cyclooctenylene, 1,3-cyclopentadienylene, 1,3-cyclohexadienylene, 1,4-cyclohexadienylene, 1,3-cycloheptadienylene, 1,3,5-cycloheptatrienylene, 1,3,5,7-cyclooctatetraenylene and the like. The cycloalkenylene group also can be optionally substituted (a “substituted cycloalkenylene”) with an “alkyl group substituent”, as defined herein.

“Alkoxyl” or “alkoxyalkyl” refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl and/or substituted alkyl as previously described.

“Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group and the term “carboxylate” refers to an anion formed from a carboxyl group, i.e., —COO⁻¹.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “phosphate” refers to phosphorous oxoacids, including the —H₂PO₃ and —H₃PO₄ groups

The term, “thio” refers to a compound described previously herein wherein a carbon or oxygen atom is replaced by a sulfur atom.

The term “sulfate” refers to the —SO₄ group.

When a named atom or group is defined as being “absent,” the named atom is replaced by a direct bond or a hydrogen.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

A named “R₁”, “R₂”, “R₃”, “A₁”, “A₂”, and “B” group will generally have the structure that is recognized in the art as corresponding to a group having that name, unless specified otherwise herein. For the purposes of illustration, certain representative “R,” “X,” and “Y” groups as set forth above are defined below. These definitions are intended to supplement and illustrate, not preclude, the definitions that would be apparent to one of ordinary skill in the art upon review of the present disclosure.

The term “binding” or “bind” as used herein, refers to the noncovalent association of one or more molecules with another molecule. The molecules involved in binding can be small molecules produced by organic synthesis, portions of DNA or RNA molecules, proteins or combinations thereof. Thus, “binding” can involve hybridization or more general hydrogen bonding and/or other non-covalent interactions, such as ionic bonding, hydrophobic interactions, interactions based on Van der Waals forces or London dispersion forces, and dipole-dipole interactions.

As used herein, the term “modulate” means an increase, decrease, or other alteration of any, or all, chemical and biological activities or properties of a wild-type or mutant polypeptide, such as a relaxase polypeptide. The term “modulation” as used herein refers to both upregulation (i.e., activation or stimulation) and downregulation (i.e. inhibition or suppression) of a response.

The term “inhibitor” refers to a chemical substance that inactivates or decreases the biological activity of a polypeptide, such as a relaxase polypeptide enzyme.

As used herein, the terms “effective amount” and “therapeutically effective amount” are used interchangeably and mean a dosage sufficient to provide treatment for the disease state being treated. This can vary depending on the patient, the disease and the treatment being effected.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

### III. MECHANISM OF RELAXASE-MEDIATED CONJUGATIVE DNA TRANSFER

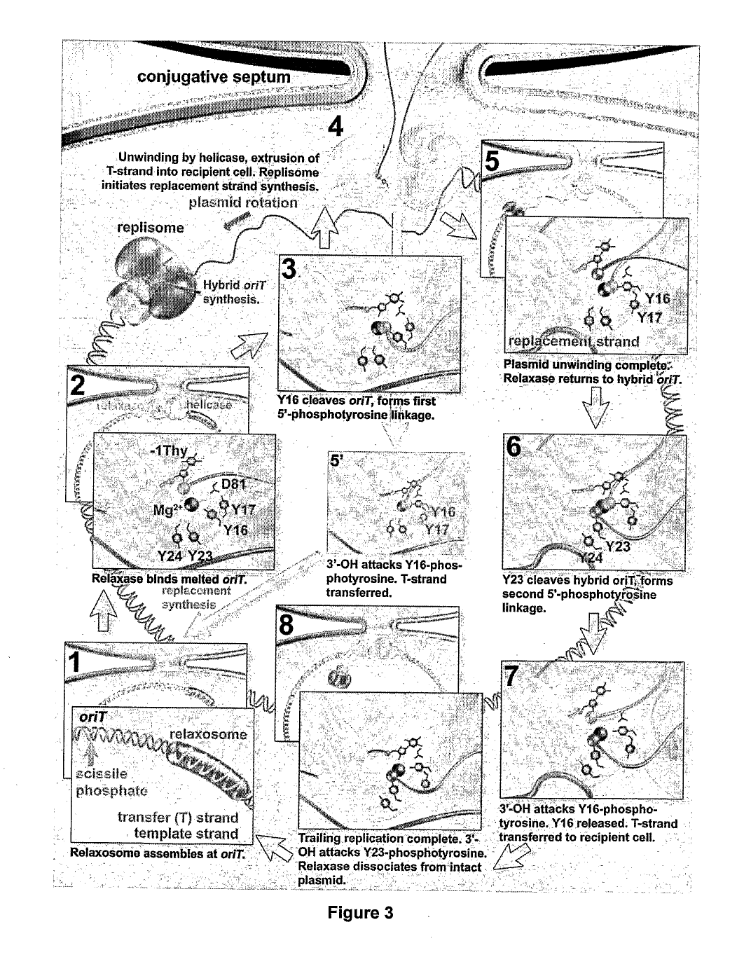

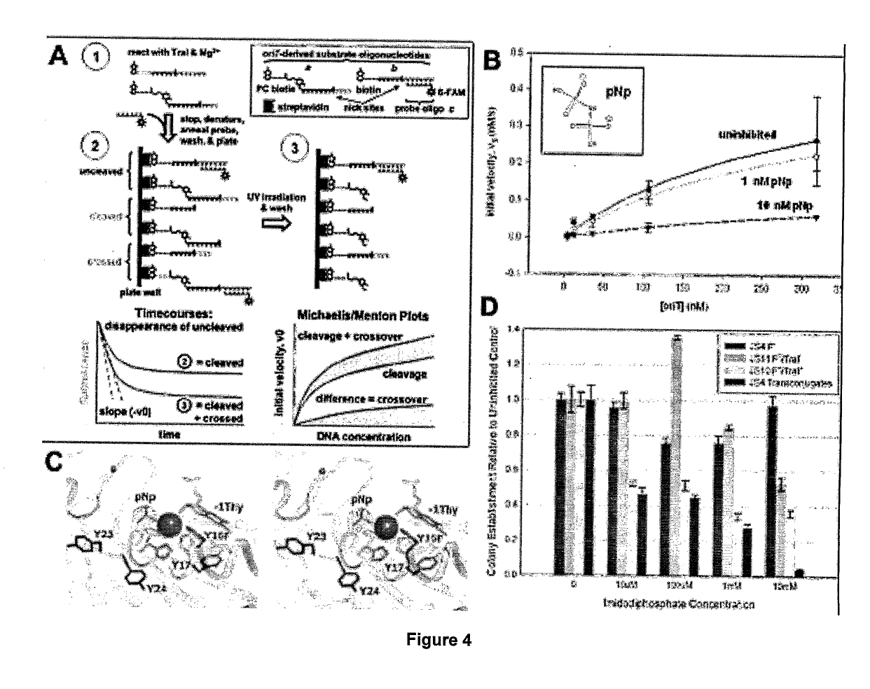

The comprehensive mechanism for conjugative DNA transfer presented in FIG. 3 provides the first detailed framework in which to examine the concerted catalytic and non-catalytic actions of pump-type relaxases. In particular, this mechanism provides two clear observations about the relaxase active site: two phosphate-containing intermediates are capable of binding simultaneously, and precise structural changes are necessary to position side chains for catalysis. Structural and functional data presented herein in the Examples are consistent with the first of these observations: a bis-phosphonate compound potently inhibits the catalytic action of TraI (FIG. 4).

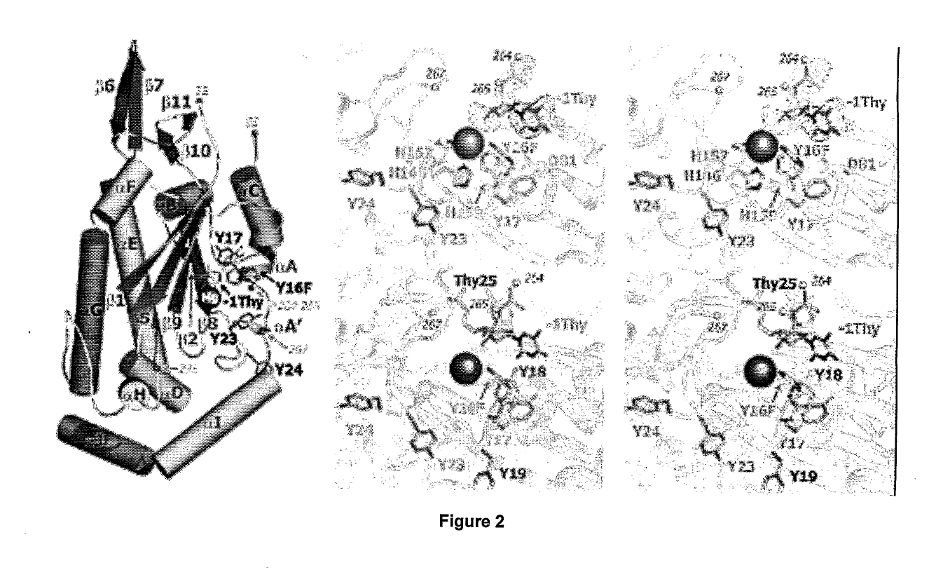

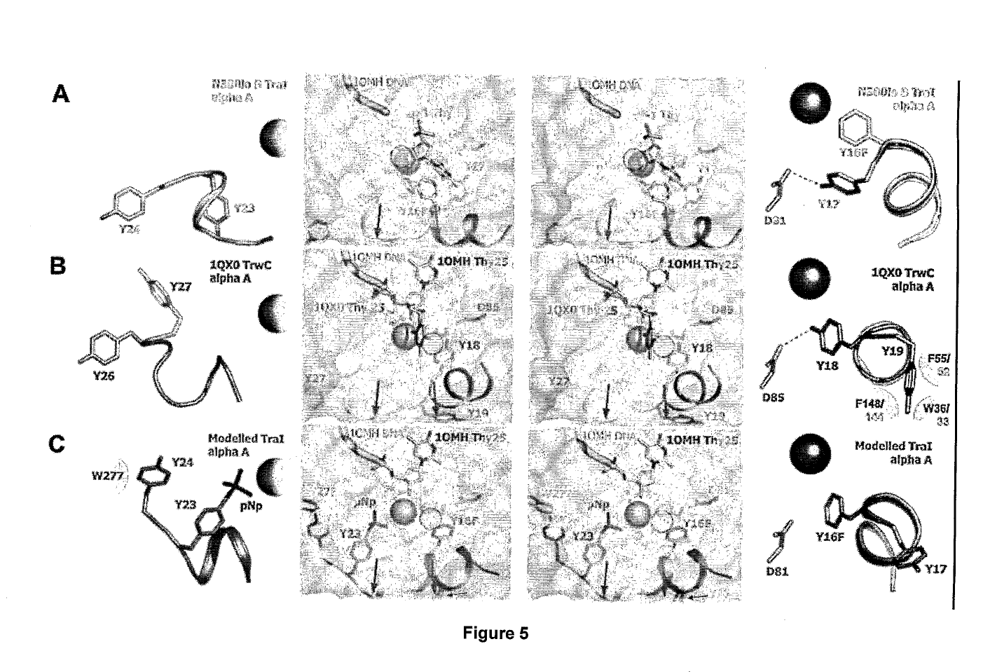

The second observation provided by the mechanism in FIG. 3 is that the TraI tyrosines undergo precise step-wise changes in position to align the catalytic side chains Y16 and Y23 for concerted nucleophilic attacks. Three states are proposed for these tyrosines (FIG. 5); two have been observed structurally, and the third can be modeled based on the structure of TraI N300-DNA nucleotide-pNp ternary complex structure described herein (FIG. 5). Initially, Tyr-16 is aligned for an in-line attack on the scissile phosphodiester linkage located between the oriT-1 T and +1 G nucleotides (FIG. 5A; Step 3 in FIG. 3). Note that an in-line attack is observed in the N300-DNA nucleotide structure presented in FIG. 2. In this orientation, Tyr-17 forms a hydrogen bond with the side chain of Asp-81, while tyrosines 23 and 24 are swung 12.0 and 17.6 Å away (tyrosine hydroxyl to magnesium distance), respectively, from the catalytic site. After F plasmid unwinding, the 5′-phosphotyrosine intermediate on Tyr-16 rotates over and docks adjacent to Asp-81, in the position occupied previously by Tyr-17 (FIG. 5B; Step 5 in FIG. 3). The shift is generated by the rotation of αA by one-third of a turn about its helical axis. This rotation places the side chain of Tyr-17 in a hydrophobic pocket formed by Trp-33, Phr-52 and Phe-144 (residues completely conserved in the pump-type relaxases; FIG. 5B). The analogous tyrosine was observed in this position in the TrwC-DNA complex. Further, the ssDNA bound in the TrwC-DNA structure represents the position of the 3′-hydroxyl generated by the second transesterification reaction (Step 6 in FIG. 3). The concerted αA shift would place the Y16-phosphotyrosine group in position for attack by this 3′-hydroxyl. In addition, the location of the scissile phosphotyrosine linkage between the Mg²⁺ atom and D81 would polarize the bond for this attack, effectively making Y16 a better leaving group.

Tyrosines 23 and 24 have been observed to dock well away from the catalytic site in the TraI N300, N330 and TrwC structures reported to date (FIGS. 5A, B) (Datta et al., 2003; Guasch et al., 2003). If TraI αA is modeled as a helix that continues through Tyr-24, however, the side chain of Tyr-23 rotates up into close proximity to the active site. Remarkably, the hydroxyl oxygen on this side chain superimposes on an oxygen of the bound relaxase inhibitors (e.g., pNp phosphonate) described herein (FIG. 5C; see also FIG. 4B). This is the active orientation of the second catalytic tyrosine when involved in a covalent 5′-phosphotyrosine linkage (Steps 6-8 in FIG. 3). Further, the Tyr-24 side chain is found in this model to be in van der Waals contact with the side chain of Trp-277, which is conserved in the known pump relaxases. Thus, the rotation and lengthening of αA in TraI provides the concerted motion necessary for the enzyme to achieve three distinct but catalytically necessary orientations within its active site. By doing so, the enzyme is able to perform the two sequential DNA nicking and religation reactions needed when conjugative DNA transfer is linked to replicative strand replacement. The series of conserved interactions the TraI tyrosine side chains form with hydrophobic side chains also explains why the aromatic ring character of these residues is critical to enzyme function (see Table II).

Low levels of enzyme activity have been observed in vitro even after TraI/TrwC Y16/18F mutations (Grandoso et al., 2000). This can be a side effect of active site flexibility, which would allow a proximal intact tyrosine to rotate into the active site to perform a remedial catalytic event. Such a cleavage event likely occurred during the growth of the crystals described here, which appeared after more than 30 days. The 9-mer ssDNA oligonucleotide was nicked either by another tyrosine in this Y16F mutant (i.e., Y17, Y23 or Y24), or by a water molecule activated by the bound 2+ metal ion, as discussed below.

The pump-type TraI is similar in overall sequence and fold to both pilot-type relaxases (e.g., TraI of plasmid RP4, MobA of RSF1010, TraA of Agrobacterium pTi) and to simple viral enzymes vital to phage rolling circle replication (e.g., Rep of mammalian Adeno Associated Virus serotype 5, Epstein-Barr Virus, Hepatitis Delta Virus and of Tobacco Yellow Leaf Curl Virus), and therefore relaxase inhibitors effective for modulating pump-type relaxases such as TraI can also prove useful against pilot-type relaxases and viral rolling circle replication enzymes. However, pump-type relaxases also exhibit two unique features. First they contain a cluster of two to five tyrosine residues near their N-termini. Second, they contain a highly efficient and processive 5′→3′ helicase fused to their C-termini (Abdel-Monem et al., 1976; Dash et al., 1992; Lahue and Matson, 1988). A cladogram was generated to examine the evolutionary relationship between pump- and pilot-type relaxases (Tables IV and V). Members of the IncQ family of pilot relaxases, which contain only a single catalytic tyrosine, were found to be distantly related to the pump-type relaxases like F plasmid TraI. A family of Rhisobiales plant-infecting bacteria are intermediate between the pump and pilot relaxases, as they combine a single-tyrosine pilot relaxase with a helicase domain (Table IV). These intermediate enzymes can unwind the transferred plasmid in the donor cell before piloting the 5′-end of the DNA into the recipient cell.

Pump-type relaxases contain the fused helicase domain as well as a duplication of the catalytic tyrosines at their N-termini (Table IV). Thus, as appreciated herein for the first time, these non-piloting relaxases could orchestrate efficient and processive conjugative DNA transfer without physically chaperoning the DNA into the recipient cell. One key advantage of this arrangement is that the same relaxase, because it remains within the donor cell, could mediate the transfer of many copies of a conjugative plasmid. The catalytic tyrosines defined above for F plasmid TraI (Y16 and Y23) are conserved in all the pump-type relaxase sequences examined; the non-catalytic tyrosines are replaced, in some cases, by phenylalanines or hydrophobic side chains (Table IV). Two catalytic tyrosines would allow the pump relaxases to perform the step-wise DNA cleavage and religation events proposed in FIG. 4 within the donor cell, which would remove the need for the pump relaxases to pilot. In support of the proposed mechanism, the pump-type relaxases contain conserved residues equivalent to the following TraI side chains proposed above to be critical to enzyme function: Asp-81, neutral amino acids coordinating the Mg²⁺ (typically histidines, but glutamines are also observed), a hydrophobic pocket for Tyr-17 (Trp-33, Phe-52 and Phe-144), and Trp-277 proposed for Tyr-24 positioning. Thus, the evolutionary relationship between the pump and pilot relaxases, and within the pump relaxase family itself, completely supports the mechanistic model for conjugative DNA transfer outlined in FIG. 3, which was derived from a combination of structural, functional and mutagenesis data disclosed herein.

The Mg²⁺ ion coordination in the TraI relaxase is atypical of protein-Mg complexes. As of September 2003, 518 of 658 (78.7%) Protein Data Bank (PDB) magnesium-containing crystal structures of ≦2.5 angstrom resolution displayed magnesium binding sites having at least one acidic side chain (http://tanna.bch.ed.ac.uk/, (Harding, 2004)). In pump-type relaxases like TraI, however, only neutral residues coordinate ions bound at the active site. These observations support the conclusion that the 2+ charge on the bound metal ion plays a role in the catalytic cycle of the pump relaxases (see Table II). In Type restriction endonucleases (T2REases), bound magnesium apparently aids in the deprotonation of a water molecule from bulk solvent in order to hydrolyze a phosphodiester bond (Pingoud et al., 2005; Pingoud and Jeltsch, 2001). However, this is not a particularly favorable reaction, as the pKa of water is ˜15 and the full magnesium +2 charge is not brought to bear due to chelation by acidic residues. The type II DNA Topoisomerases (Topo IIs), which use acidic residues for Mg-chelation, appear to employ a somewhat more favorable mechanism, as these enzymes employ a tyrosine nucleophile with a pKa of 10, far closer to physiological pH. In contrast, the neutral histidine Mg-chelating residues in relaxases maintain a full divalent positive charge, and thus appear ideally suited to deprotonate tyrosine hydroxyls for nucleophilic attack. Indeed, of the remaining 140 magnesium-containing structures identified in the PDB survey above, 133 (95%) bind polyphosphate ligands (nucleotide di- and triphosphates, nicotinamide adenine dinucleotide and its derivatives, etc.). These 133 structures fell into 24 clusters with ≧30% sequence identity within each cluster, and visual inspection of representative structures revealed that two phosphate groups bound to the magnesium in all structures. Thus, these structural observations support the conclusion that a 2+ magnesium charge favorably allows the presence of two phosphotyrosine intermediates simultaneously in the pump-type relaxase active site, as predicted by the comprehensive mechanism in FIG. 3.

While it is less favorable than the activation of a tyrosine side chain, a 2+ metal ion is capable of activating a water molecule for nucleophilic attack. This likely occurred during the growth of the crystals described herein. A tyrosine other than Y16 might have nicked the ssDNA and the resulting phosphotyrosine intermediate released by an activated water molecule. Alternatively, water activated for nucleophilic attack by the 2+ magnesium might have performed the ssDNA nicking event by itself, analogous to the T2REases. The role of activated water molecules in vivo, which would lead to unproductive DNA cleavage and transfer events, could be limited in the full-length enzyme in its natural context by conformational changes that protect substrates and catalytic intermediates within the relaxase active site.

### IV. COMPOUNDS

IV.A. Relaxase Modulating Compounds

Disclosed herein is a class of compounds that modulate the activity of relaxase enzymes. In some embodiments, the compounds bind to and inhibit the activity of a relaxase enzyme. In some embodiments, activity inhibited by the compounds includes polynucleotide cleavage and/or polynucleotide religation by the relaxase.

By inhibiting the activity of a relaxase enzyme, the compounds of the presently disclosed subject matter can be utilized to inhibit bacterial conjugation, which in turn can reduce the spread of antibiotic resistance genes and virulence factors between bacteria by conjugation. Further, the compounds disclosed herein can be utilized as antimicrobial agents, including bactericidal agents by directly killing bacteria (for example, due to relaxase binding activity) and/or antiviral agents. For example, certain virus strains replicate using proteins sharing homology with relaxase enzymes, such as for example viral Rep proteins.

In some embodiments, the compounds disclosed herein have a net negative charge, and in some embodiments, the compounds have a −2 charge. In some embodiments the compound comprises a phosphate, carboxylate, sulfate, or nitro moiety, which can in some embodiments be a bis-moiety (e.g., a bis-phosphate moiety).

In some embodiments, the relaxase modulating compound is a compound having the structure of Formula (I):

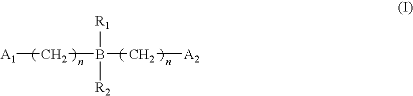

wherein:

n is an integer from 0 to 4;

A₁ and A₂ are independently selected from the group consisting of H, hydroxyl, alkyl, substituted alkyl, cycloalkyl, substituted cycloalkyl, aryl, substituted aryl, aralkyl, substituted aralkyl, phosphate, carboxylate, sulfate, and nitro, provided that at least one of A₁ or A₂ is phosphate, carboxylate, sulfate, or nitro;

B is selected from the group consisting of N, alkylene, substituted alkylene, cycloalkylene, substituted cycloalkylene, cycloalkenylene, substituted cycloalkenylene, arylene, and substituted arylene; and

R₁ and R₂ can each be present or absent and are independently selected from the group consisting of H, hydroxyl, halo, alkyl, substituted alkyl, cycloalkyl, substituted cycloalkyl, aryl, substituted aryl, aralkyl, and substituted aralkyl, or a pharmaceutically acceptable salt thereof.

In some embodiments, the relaxase modulating compound of Formula I is selected from the group consisting of imidodiphosphate, methylenediphosphonate, etidronate, clodronate, pamidronate, alendronate, neridronate, iminobis, N-(2-hydroxyethyl)iminobis, glyphosine, 1,2-bis(dimethoxyphosphoryl)benzene, dichloromethylenediphosphonate, and SR12813 (3,5-di-tert-butyl-4-hydroxystyrene-β,β-diphosphonic acid tetraethyl ester). Further, in some embodiments, the relaxase modulating compound of Formula I has a structure selected from the group consisting of:

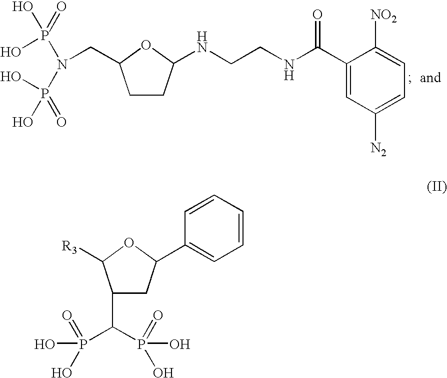

wherein:

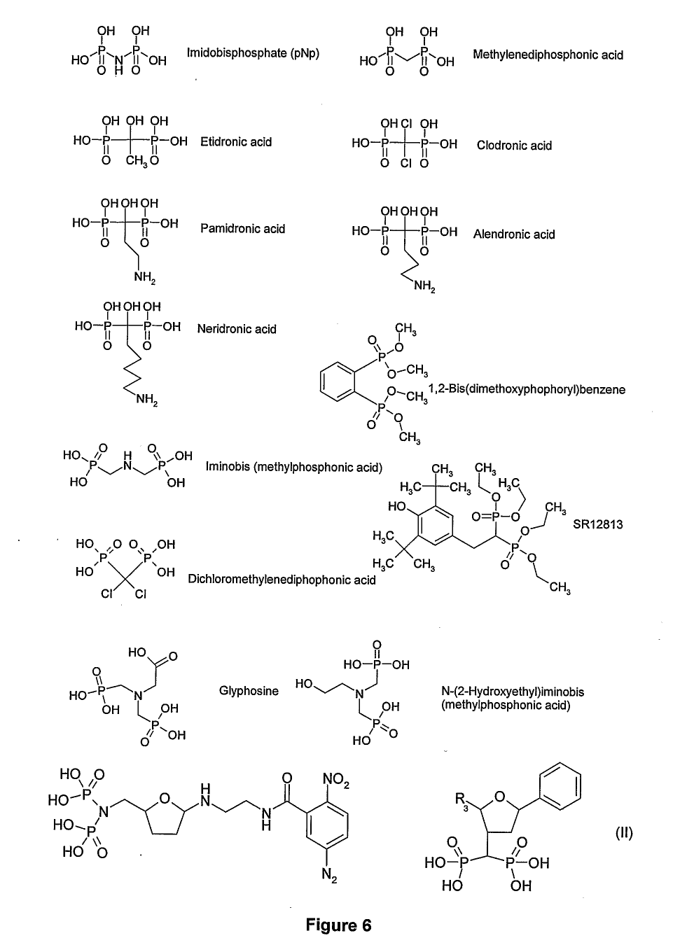

R₃ is selected from the group consisting of H, hydroxyl, alkyl, substituted alkyl, cycloalkyl, substituted cycloalkyl, aryl, substituted aryl, aralkyl, and substituted aralkyl. Exemplary structures of relaxase modulating compounds are also disclosed in FIG. 6.

IV.B. Prodrugs

In representative embodiments, compounds disclosed herein are prodrugs. A prodrug means a compound that, upon administration to a recipient, is capable of providing (directly or indirectly) a compound of the presently disclosed subject matter or an inhibitorily active metabolite or residue thereof. Prodrugs can increase the bioavailability of the compounds of the presently disclosed subject matter when such compounds are administered to a subject (e.g., by allowing an orally administered compound to be more readily absorbed into the blood) or can enhance delivery of the parent compound to a biological compartment (e.g., the brain or lymphatic system) relative to a metabolite species, for example.

IV.C. Pharmaceutically Acceptable Salts

Additionally, the active compounds can be administered as pharmaceutically acceptable salts. Such salts include the gluconate, lactate, acetate, tartarate, citrate, phosphate, borate, nitrate, sulfate, and hydrochloride salts. The salts of the compounds described herein can be prepared, in general, by reacting two equivalents of the base compound with the desired acid, in solution. After the reaction is complete, the salts are crystallized from solution by the addition of an appropriate amount of solvent in which the salt is insoluble. In a particular embodiment, the pharmaceutically acceptable salt is a hydrochloride salt.

### V. PHARMACEUTICAL FORMULATIONS

The presently disclosed compounds, pharmaceutically acceptable salts thereof, prodrugs corresponding thereto, and the pharmaceutically acceptable salts thereof, are all referred to herein as “active compounds.” That is, these are compounds that can modulate an enzymatic activity of a relaxase polypeptide. Pharmaceutical formulations comprising the aforementioned active compounds are also provided herein. These pharmaceutical formulations comprise active compounds as described herein, in a pharmaceutically acceptable carrier. Pharmaceutical formulations can be prepared for oral, intravenous, or aerosol administration as discussed in greater detail below. Also, the presently disclosed subject matter provides such active compounds that have been lyophilized and that can be reconstituted to form pharmaceutically acceptable formulations for administration, as by intravenous or intramuscular injection.

The therapeutically effective dosage of any specific active compound, the use of which is in the scope of embodiments described herein, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level can restrict intravenous dosages to a lower level such as up to about 10 mg/kg, with all weights being calculated based upon the weight of the active base, including the cases where a salt is employed. A dosage from about 0.01 mg/kg to about 100 mg/kg can be employed for oral administration. Typically, a dosage from about 0.1 mg/kg to 30 mg/kg can be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once or twice per day for a period of two to three weeks or until the condition is essentially controlled. Lower doses given less frequently can be used prophylactically to prevent or reduce the incidence of recurrence of the condition, e.g., a microbial infection.

In accordance with the present methods, pharmaceutically active compounds as described herein can be administered orally as a solid or as a liquid, or can be administered intramuscularly or intravenously as a solution, suspension, or emulsion. Alternatively, the compounds or salts can also be administered by inhalation, intravenously or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical formulations suitable for intravenous or intramuscular injection are further embodiments provided herein. The pharmaceutical formulations comprise an active compound as disclosed herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. Of course, the dispensing is preferably done by an aseptic method. Sterilized closures can then be placed on the vials and, if desired, the vial contents can be lyophilized.

In addition to the active compounds, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain anti-microbial preservatives. Useful anti-microbial preservatives include methylparaben, propylparaben, and benzyl alcohol. The anti-microbial preservative is typically employed when the formulation is placed in a vial designed for multi-dose use. The pharmaceutical formulations described herein can be lyophilized using techniques well known in the art.

In yet another aspect of the subject matter described herein, there is provided an injectable, stable, sterile formulation comprising an active compound disclosed herein in a unit dosage form in a sealed container. The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt. When the compound or salt is substantially water-insoluble, a sufficient amount of emulsifying agent, which is physiologically acceptable, can be employed in sufficient quantity to emulsify the compound or salt in an aqueous carrier. One such useful emulsifying agent is a phosphatidylcholine.

Other pharmaceutical formulations can be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidylcholines, such as for example lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations containing the active compounds disclosed herein can be lyophilized to produce a lyophilizate, which can be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used in the present specification, the term “water-soluble” is meant to define any composition that is soluble in water in an amount of about 50 mg/mL, or greater. Also, as used in the present specification, the term “water-insoluble” is meant to define any composition that has solubility in water of less than about 20 mg/mL. For certain applications, water-soluble compounds or salts can be desirable whereas for other applications water-insoluble compounds or salts likewise can be desirable.

### VI. THERAPEUTIC METHODS

It is clear from the data disclosed in the Examples section that relaxase inhibitors, such as for example pNp, limit conjugative DNA transfer and preferentially kill E. coli cells in a TraI-dependent manner (FIG. 4D). Note that JS10 (F+) cells, which are susceptible to relaxase inhibitors such as pNp, are not capable of undergoing mating because no F− cells are present.

Without wishing to be limited by theory, the relaxosome is likely assembled on the F plasmid even when mating has not been initiated, allowing TraI to undergo a cycle of nicking and religating at the oriT. Again, without wishing to be limited by theory, it is likely that relaxase inhibitors can interfere with this process, perhaps exposing the 3′-OH for use by the cellular replication machinery. In this case resolution of the replication reaction would be prevented allowing the formation of concatemers of ssDNA. Unchecked, this would effectively drain cellular resources and result in cell killing. It is also possible that certain relaxase inhibitors, such as a bis-phosphonate like pNp may have other cellular targets like pyrophosphotases or polymerases that enhance lethality. Thus, the relaxase inhibitors of the presently disclosed subject matter can be utilized in methods of inhibiting bacterial conjugation, as well as antimicrobials in methods of treating microbial infections, including both bacterial and viral infections.

In some embodiments of the presently disclosed subject matter, a method of inhibiting bacterial conjugation is provided, comprising contacting a bacterium having a relaxase enzyme with a relaxase dependent antibiotic. The relaxase dependent antibiotic can comprise a compound disclosed herein, capable of binding and modulating the enzymatic activity of a relaxase enzyme within the bacteria. In particular embodiments, the relaxase dependent antibiotic targets a Mob family relaxase, such as for example a TraI relaxase.

In some embodiments, the relaxase dependent antibiotic is co-administered to the bacterium with at least one additional antibiotic. The co-administration need not necessarily be at precisely the same time, but rather can if desirable be a staggered administration. For example, the relaxase dependent antibiotic can be administered sequentially before or after the administration of the additional one or more antibiotics. This therapeutic approach can reduce the spread of antibiotic resistance to the additional antibiotic between bacteria, as the relaxase dependent antibiotic can inhibit bacterial conjugation in the targeted bacteria, thereby blocking a primary mode of genetic transfer between bacteria.

In some embodiments of the presently disclosed subject matter, a method of treating a microbial infection in a subject is provided. “Treating a microbial infection”, as the phrase is used herein, refers not only to the treatment of a microbial infection already present within the subject, but also to the prophylactic administration of a compound of the presently disclosed subject matter to a subject prior to establishment of a microbial infection in the subject in order to aid in the prevention or lessening of the severity of microbial infections in the subject. In some embodiments, the method comprises administering to the subject an effective amount of a compound of Formula (I). In some embodiments, the method comprises administering to the subject an effective amount of a compound that modulates an enzymatic activity of a relaxase polypeptide. These infections can be caused by a variety of microbes, including bacteria and viruses. Otherwise normal microbial flora can in some instances also be considered a microbial infection, especially when the flora causes harm to the host organism. As previously disclosed, the antimicrobial compounds disclosed herein (e.g., compounds of Formula (I)) can exhibit binding specificity for both bacterial and/or viral relaxases.

Representative bacterial infections that can be treated or prevented by the methods of the presently disclosed subject matter can include those bacteria expressing Mob relaxase polypeptides, such as for example TraI relaxase polypeptides. Exemplary bacterial infections that can be treated with the presently disclosed methods include infections caused by bacteria such as, for example, Escherichia (e.g., E. coli), Salmonella, Shigella, Actinobacillus, Porphyromonas, Staphylococcus, Bordetella, Yersinia, Haemophilus, Streptococcus, Chlamydophila, Alliococcus, Campylobacter, Actinomyces, Neisseria, Chlamydia, Treponema, Ureaplasma, Mycoplasma, Mycobacterium, Bartonella, Legionella, Ehrlichia, Listeria, Vibrio, Clostridium, Tropheryma, Actinomadura, Nocardia, Streptomyces, and Spirochaeta.

In addition, relaxases are similar in sequence, structure, and mechanism to replication initiator (Rep) proteins required for certain viruses, including human viruses, that use rolling circle replication (RCR). Representative viral infections that can be treated or prevented by the methods of the present subject include those viruses that replicate using a rolling-circle-replication method involving a viral relaxase. Exemplary viral infections that can be treated include, but are not limited to those infections caused by adeno associated viruses, including for example Adeno Associated Virus serotype 5, Epstein-Barr Virus, Hepatitis delta Virus, and certain tumor viruses.

Active compounds utilized with the methods disclosed herein can modulate an enzymatic activity of a relaxase polypeptide. In some embodiments, modulating an enzymatic activity of a relaxase polypeptide is inhibiting the enzymatic activity. When active compounds are contacted with bacterial relaxases, inhibiting relaxase activity includes, for example, inhibiting polynucleotide cleavage and/or polynucleotide religation activities of the relaxase. When active compounds are contacted with viral relaxases, inhibiting relaxase activity includes, for example, inhibiting viral nucleic acid replication activities of the relaxase.

These active compounds, as set forth above, include compounds having a net negative charge (for example, but not limited to −1, −2, and −3). In some embodiments, the net negative charge results from a phosphate, carboxylate, sulfate, or nitro moiety, and in some embodiments, these are bis-moieties (e.g., bis-phosphate, bis-carboxylate, bis-sulfate, and bis-nitro). In some embodiments, the active compounds include compounds having a structure of Formula (I), their corresponding prodrugs, and pharmaceutically acceptable salts of the compounds and prodrugs.

As set forth in detail above, compounds of Formula (I) are defined as having a structure as follows:

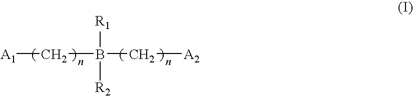

wherein:

n is an integer from 0 to 4;

A₁ and A₂ are independently selected from the group consisting of H, hydroxyl, alkyl, substituted alkyl, cycloalkyl, substituted cycloalkyl, aryl, substituted aryl, aralkyl, substituted aralkyl, phosphate, carboxylate, sulfate, and nitro, provided that at least one of A₁ or A₂ is phosphate, carboxylate, sulfate, or nitro;

B is selected from the group consisting of N, alkylene, substituted alkylene, cycloalkylene, substituted cycloalkylene, cycloalkenylene, substituted cycloalkenylene, arylene, and substituted arylene; and

R₁ and R₂ can each be present or absent and are independently selected from the group consisting of H, hydroxyl, halo, alkyl, substituted alkyl, cycloalkyl, substituted cycloalkyl, aryl, substituted aryl, aralkyl, and substituted aralkyl, or a pharmaceutically acceptable salt thereof.

In some embodiments, the active compound of Formula I is selected from the group consisting of imidodiphosphate, methylenediphosphonate, etidronate, clodronate, pamidronate, alendronate, neridronate, iminobis, N-(2-hydroxyethyl)iminobis, glyphosine, 1,2-bis(dimethoxyphosphoryl)benzene, dichloromethylenediphosphonate, and SR12813 (3,5-di-tert-butyl-4-hydroxystyrene-β,β-diphosphonic acid tetraethyl ester). Further, in some embodiments, the active compound of Formula I has a structure selected from the group consisting of:

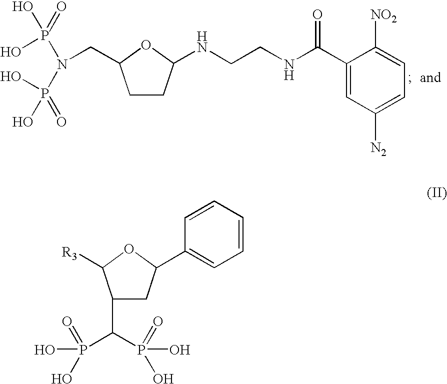

wherein:

R₃ is selected from the group consisting of H, hydroxyl, alkyl, substituted alkyl, cycloalkyl, substituted cycloalkyl, aryl, substituted aryl, aralkyl, and substituted aralkyl. Exemplary structures of relaxase modulating compounds are disclosed in FIG. 6

VI.A. Subjects

Further with respect to the therapeutic methods of the presently disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

VI.B. Doses

The term “effective amount” is used herein to refer to an amount of the active compound (e.g., a composition comprising a compound that modulates an enzymatic activity of a relaxase polypeptide) sufficient to produce a measurable biological response (e.g., a reduction in an enzymatic activity of a relaxase polypeptide, such as for example a reduction in polynucleotide cleavage and/or polynucleotide religation (e.g., crossover religation) activity of the relaxase). Actual dosage levels of active ingredients in a active compound of the presently disclosed subject matter can be varied so as to administer an amount of the active compound(s) that is effective to achieve the desired therapeutic response for a particular subject and/or application. The selected dosage level will depend upon a variety of factors including the activity of the active compound, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art of medicine.

For administration of an active compound as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., (1966)). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (Freireich et al., (1966)). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For oral administration, a satisfactory result can be obtained employing an active compound in an amount ranging from about 0.01 mg/kg to about 100 mg/kg and preferably from about 0.1 mg/kg to about 30 mg/kg. A preferred oral dosage form, such as tablets or capsules, will contain the therapeutic compound in an amount ranging from about 0.1 to about 500 mg, preferably from about 2 to about 50 mg, and more preferably from about 10 to about 25 mg.

For parenteral administration, the active compound can be employed in an amount ranging from about 0.005 mg/kg to about 100 mg/kg, preferably about 10 to 50 or 10 to 70 mg/kg, and more preferably from about 10 mg/kg to about 30 mg/kg.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

VI.C. Routes of Administration

Suitable methods for administering to a subject an active compound in accordance with the methods of the presently disclosed subject matter include but are not limited to systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

The particular mode of active compound administration used in accordance with the methods of the present subject matter depends on various factors, including but not limited to the vector and/or drug carrier employed, the severity of the condition to be treated, and mechanisms for metabolism or removal of the drug following administration.

VI 1. Assays

Also disclosed herein are novel assays, developed to measure catalytic kinetic time courses of multisubstrate enzymes, including polynucleotide specific enzymes, such as for example relaxases, which was previously not possible using standard polyacrylamide gel electrophoresis methods. Other multisubstrate enzymes that can be analyzed using the assay include, but are not limited to, type IA topoisomerases, recombinases, integrases, and transposases.

The assay method provided herein for measuring kinetic time courses of polynucleotide specific enzymes can comprise the following steps. A multifunctional polynucleotide-specific enzyme, a first substrate polynucleotide, and a second substrate polynucleotide are provided. The multifunctional polynucleotide-specific enzyme can be an enzyme capable of exhibiting multiple types of enzymatic activity, depending upon the substrate, reaction conditions, etc.

The first substrate polynucleotide comprises a capture tag linked to a first end of the first polynucleotide, an enzyme recognition polynucleotide sequence, and a label linked to a second end of the first polynucleotide. The second substrate polynucleotide comprises an enzyme recognition polynucleotide sequence and a cleavable capture tag linked to an end of the second polynucleotide. The enzyme to be studied, the first polynucleotide and the second polynucleotide are incubated for a time sufficient to permit the enzyme to react with the first polynucleotide and the second polynucleotide. The first polynucleotide and the second polynucleotide are captured to a capture affinity molecule having binding affinity for both the capture tag and the cleavable capture tag, wherein the capture affinity molecule is bound to a substrate. The substrate is washed to remove uncaptured molecules. A first kinetic time course of the enzyme is determined based on a measured change in an amount of the label bound to the substrate over a time course. The cleavable capture tag is cleaved, thereby releasing the second polynucleotide from the substrate. A second kinetic time course of the enzyme is determined based on a measured change in an amount of the label bound to the substrate before and after cleavage of the cleavable capture tag over a time course.

In particular embodiments of the method, the multifunctional polynucleotide-specific enzyme analyzed is a relaxase enzyme, such as for example a Mob relaxase enzyme (e.g., TraI).

The first and second substrate polynucleotides can comprise sequences for which the enzyme has binding specificity (enzyme recognition polynucleotide sequences). For example, in some embodiments, a relaxase enzyme is analyzed, and the substrate polynucleotides can comprise a bacterial oriT sequence, which is specifically recognized and can be bound and cleaved by the relaxase enzyme. The ability of the relaxase enzyme to cleave both substrate polynucleotides further makes available the cleaved polynucleotide sites to the relaxase enzyme for a second measurable enzymatic activity: polynucleotide religation, wherein a polynucleotide strand crossover event occurs and the cleaved nucleotide ends from each substrate polynucleotide are religated to opposite substrate polynucleotides at the site of original cleavage. The two separate enzymatic events of cleavage and crossover religation can be distinguished and measured because the first substrate polynucleotide comprises a capture tag and the second substrate polynucleotide comprises a cleavable capture tag. Further, the first substrate polynucleotide comprises a label, whereas the second substrate polynucleotide does not.

As used herein, a capture tag is any compound that can be associated with a compound of interest, and which can be used to separate compounds associated with the capture tag from those not associated with the capture tag. Exemplary capture tags include, but are not limited to ligands, haptens, oligonucleotides, antibodies, and lipophilic molecules. The capture tag can be cleavable or non-cleavable. One preferred form of capture tag is a compound, such as a ligand or hapten, which binds to or interacts with another compound, referred to herein as a capture affinity molecule, such as a ligand-binding molecule or an antibody. In some embodiments, such an interaction between the capture tag and the capture affinity molecule is a specific interaction, such as between a hapten and an antibody or a ligand and a ligand-binding molecule.

In some embodiments, the capture tags include biotin, which can be incorporated into nucleic acids (Langer et al., 1981) and captured using streptavidin or biotin-specific antibodies (i.e., the capture affinity molecule). Both cleavable (e.g., photocleavable) and non-cleavable forms of biotin can be utilized to distinguish, for example, between the first and second substrate polynucleotides.

Exemplary haptens for use as capture tags further include digoxigenin, Protein A, Protein G. Further, many compounds for which a specific antibody is known, or for which a specific antibody can be generated, can also be used as capture tags. Such capture tags can be captured by antibodies which recognize the compound. Antibodies can also be useful as capture tags, which can be obtained commercially or produced using well established methods.

Another capture tag useful with the presently disclosed methods is one that can be used in an anti-antibody method. Such anti-antibody-antibodies and their use are well known. In these methods, the hapten for the anti-antibody is an antibody. For example, anti-antibody-antibodies that are specific for antibodies of a certain class (for example, IgG, IgM), or antibodies of a certain species (for example, anti-rabbit antibodies) are commonly used to detect or bind other groups of antibodies. Thus, an antibody to the capture tag can be reacted with the capture tag and the resulting antibody:capture tag:oligomer complex can be purified by binding to an antibody for the antibody portion of the complex.

As used herein, the terms “label” and “labeled” refer to the attachment of a moiety, capable of detection by spectroscopic, radiologic, or other methods, to a molecule to be tracked, such as for example a substrate polynucleotide. The label can be attached directly to the molecule, or indirectly by attachment to a probe molecule (e.g., a probe oligonucleotide), which in turn has binding specificity for the molecule to be tracked. Thus, the terms “label” or “labeled” refer to incorporation or attachment, optionally covalently or non-covalently, of a detectable marker into a molecule, such as a polynucleotide. Various methods of labeling polynucleotides are known in the art and can be used. Examples of labels for polynucleotides include, but are not limited to, the following: radioisotopes, fluorescent labels, heavy atoms, enzymatic labels or reporter genes, chemiluminescent groups, biotinyl groups, and predetermined polynucleotide sequences recognized by a secondary reporter. In some embodiments, labels are attached by spacer arms of various lengths to reduce potential steric hindrance.

In particular embodiments of the methods, fluorescent labels are used. Representative fluorescent labels that can be utilized include, but are not limited to fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3, 5, and 7; phycoerythrin; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives; pyrenes; pyridyloxazole derivatives; dapoxyl dyes; Cascade Blue and Yellow dyes; benzofuran isothiocyanates; sodium tetrafluorophenols; and 4,4-difluoro-4-bora-3a,4a-diaza-s-indacene. The excitation wavelength can vary for these compounds.

In some embodiments, the first and second kinetic times courses that can be measured are kinetic time courses relating to separate and distinct enzymatic activities of the enzyme being studied. Representative kinetic time course evaluations include determining maximum velocity values (Vₘₐₓ) and Michaelis constants (Kₘ).

FIG. 4A provides an exemplary setup for a particular reaction, showing the various specific polynucleotides and types of labels attached to each polynucleotide. The representative assay method for measuring multiple kinetic time courses of a multifunctional polynucleotide-specific enzyme can be utilized to measure kinetic time courses for relaxase enzymes. In particular, as disclosed in FIG. 4A and also in the Examples, the representative assay can be utilized to measure the kinetics of both DNA cleavage and crossover religation catalyzed by relaxase enzymes within the same assay setup.

The presently disclosed subject matter further provides methods for assaying and selecting for compounds that can inhibit enzymatic activities of polynucleotide-specific enzymes, such as for example relaxase enzymes. In general, the assay for measuring catalytic time courses of a polynucleotide-specific enzyme is utilized to determine a change in catalytic activity of the enzyme in the presence of absence of a candidate inhibitor.

In some embodiments, the method comprises the following steps. A polynucleotide-specific enzyme is contacted with a substrate polynucleotide comprising a label in the presence of a candidate inhibitor. The enzyme and the polynucleotide are incubated together in the presence of the candidate inhibitor for a time sufficient to permit the enzyme to catalytically react with the polynucleotide. A change in an amount of the labeled polynucleotide present over time is measured, whereby the change in the amount of labeled polynucleotide correlates with an activity of the enzyme on the polynucleotide. The candidate inhibitor is selected as an inhibitor of the enzyme if the activity of the enzyme on the polynucleotide is reduced in the presence of the candidate inhibitor, as compared to a reaction in which the candidate inhibitor is absent.

In some embodiments of the method, the multifunctional polynucleotide-specific enzyme being analyzed is a relaxase enzyme, such as for example a Mob relaxase enzyme (e.g., TraI). Further, in particular embodiments of the method, the representative method set forth in FIG. 4A is utilized to measure a change in enzymatic activity of the relaxase enzyme, including for example cleavage and/or crossover religation of substrate polynucleotides.

In some embodiments, the method comprises determining the inhibition constant (Kᵢ), the mechanism of inhibition, or both, of the inhibitor on the enzyme.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods For Examples

Protein Expression and Purification

An amino-terminal 300 residue F plasmid TraI construct, bearing a tyrosine to phenylalanine mutation at position 16 (N300 Y16F), was cloned into IMPACT® vector pTYB2 (New England Biolabs, Beverly, Mass., U.S.A.) for expression as a C-terminal intein-chitin-binding domain (CBD) fusion. Protein was expressed in either E. coli BL21 (DE3)/pLysS or HMS174 (DE3)/pLysS and was purified as per the standard IMPACT® protocol. Briefly, cellular extracts were prepared and incubated with 1 mL of Chitin Resin (New England Biolabs) per liter of cell culture. The resin was washed and incubated with 50 mM dithiothreitol (DTT) overnight to cleave the relaxase from its CBD tag. The DTT laden eluent was extensively dialyzed in 20 mM NaCl and 20 mM Tris-HCl (pH 7.5). The resulting N300 Y16F was 99% pure by SDS-PAGE, and was concentrated to 3 mg/mL for crystallization in 50 mM NaCl, 10% glycerol, and 10 mM Tris-HCl (pH 7.5) prior to flash-freezing in liquid nitrogen for storage at −80° C. Protein for functional and kinetic assays was concentrated to 42.3 μM in 150 mM NaCl, 50% glycerol, and 10 mM Tris-HCl (pH 7.5) for long-term storage at −80° C.

Oligonucleotides

A 9-base single-stranded DNA oligonucleotide (9mer) derived from the F plasmid oriT (5′-GGT GT̂G GTG-3′ (SEQ ID NO: 1), where ̂ is the scissile phosphate) was synthesized for crystallization at the UNC Lineberger Comprehensive Cancer Center Nucleic Acids Core Facility. Labeled oligonucleotides for fluorescence kinetic assays were synthesized by Integrated DNA Technologies (IDT; Coralville, Iowa, U.S.A.): 5′-biotin (bio) labeled 29mer (b29; 5′-BIO-TTT GCG TGG GGT GTAG GTG CTT TTG GGT GG-3′ (SEQ ID NO: 2)); 5′-photocleavable biotin (PCbio) labeled 31 mer (PCb31eco; 5′-PCbio-GGA ATT CTT TTT GCG TGG GGT GTAG CTG CTT T-3′ (SEQ ID NO: 3)); and 5′-6-carboxyfluorescein (6-FAM™) 15mer fluorescent probe (downF; 5′-CC ACC CAA AAG CAC C-3′ (SEQ ID NO: 4)). PCb31eco and b29 are substrate molecules for cleavage and crossover derived from F plasmid oriT. PCb31 eco contains an unused EcoRI site at 5′ terminus, in case of PC biotin failure, and a G to C transversion two bases downstream (3′-) of the nick (F plasmid T-strand position 139). DownF is a fluorescent probe complementary to the downstream portion of b29. Melting temperatures as calculated with IDT OLIGOANALYZER™ 3.0 with default parameters were 0° C. for downF versus the downstream portion of PCb31eco (7 base-pairs, one mismatch) and 50.8° C. for downF versus b29 (15 base-pairs). Oligonucleotides for site directed mutagenesis were synthesized by Integrated DNA Technologies.

Crystallization and Structure Determination

N300 Y16F crystals grew in a DNA-dependent manner in 75 mM sodium nitrate, 14% w/v PEG 3350, 10 mM spermine, and 110 μM 9mer. These rods were cryoprotected via a two-second dip in 150 mM sodium nitrate, 35% w/v PEG 3350, and 10 mM spermine and flash cooled in liquid nitrogen for storage and transport. Crystals employed for the N,N-imidobisphosphonate (pNp) complex were soaked for 24 hours in 200 mM ammonium nitrate, 40% w/v PEG 3350, and 1 mM pNp and flash cooled. Rods 200×30×20 μm in size were generated by hanging drop vapor diffusion after at least 35 days and diffracted to between 2.9 Å and 3.4 Å in-house. Data sets were collected at the Advanced Photon Source (APS) at Argonne National Laboratoy (ANL), at Southeast Regional Collaborative Access Team (SER-CAT) Sector 22 Insertion Device Beamline (22-ID) and the General Medicine and Cancer Institutes Collaborative Access Team (GM/CA-CAT) Sector 23 Insertion Device Beamline (23-IDᵢₙ; for the pNp complex). Crystals were of space group of P2₁2₁2₁ and contained two protein monomers complexes in the asymmetric unit (Table I). X-ray diffraction data were indexed and scaled with the HKL2000 or MOSFLM (CCP4) (Collaborative Computing Project, 1994). Initial phases were determined by molecular replacement in Molrep (CCP4) (Collaborative Computing Project, 1994) with the apo TraI structure (Protein Data Bank accession 1P4D) as a search model. Model adjustment was completed with O (Jones et al., 1991) and σₐ-weighted electron density maps (Read, 1986), and structures were refined using torsion angle dynamics and the maximum likelihood target as implemented in CNS (Brunger, 1998). Structure figures were constructed in PyMol v0.98 (DeLano, 2002).

TABLE I
Crystallographic Statistics

| Data Set | | N300-nuc | pNp |
|---|---|---|---|
| Space Group | | P 2₁2₁2₁ | P 2₁2₁2₁ |
| Unit Cell | a [Å] | 44.9 | 44.5 |
| Dimensions | b [Å] | 88.3 | 86.3 |
| | c [Å] | 127.3 | 127.9 |
| Resolution [Å] | | 2.42-500 | 3.00-500 |
| | | (2.42-2.46) | (3.00-3.11) |
| Completeness [%] | | 97.9 (99.3) | 92.8 (72.7) |
| R_{symmetry} [%] | | 9.8 (38.2) | 13.5 (38.2) |
| Ave. Redundancy | | 5.1 (5.2) | 4.1 (3.9) |
| Ave. I/Sigma | | 9.6 | 8.6 |
| R_{factor} [%] | | 21.3 | 21.9 |
| R_{free} [%] | | 27.0 | 31.3 |
| rms Deviations | bonds [Å\| | 0.0066 | 0.0084 |
| | angles [°] | 1.22 | 1.28 |
| Model Atoms | protein | 4142 | 4132 |
| | water | 157 | 0 |
| | ligands | 22 | 26 |

Functional Assays

Both wild-type and mutant TraI proteins (either full length protein or TraI-N300) were examined in oligonucleotide cleavage (DNA nicking), strand transfer (DNA religation) and liquid mating (DNA transfer) assays. The oligonucleotide cleavage reaction mixture (10 μl) contained 50 mM Tris-HCl (pH 7.5), 150 mM NaCl, 6 mM MgCl₂, 20% glycerol, 1 pmol 5′-end labeled 22-mer, and 1 pmol TraI (unless otherwise stated). Reactions were assembled at room temperature and incubated at 37° C. for 20 minutes. Reactions were stopped by the addition of SDS to 0.2%, and incubation was continued at 37° C. for 10 minutes. Ten μl 85% formamide, 50 mM EDTA, 0.1% dyes were added to the reaction, the products were denatured at 100° C. for 3 minutes and analyzed on a 16% polyacrylamide, 8 M urea denaturing gel. The gels were electrophoresed at 25 watts in 1×TBE (90 mM Tris-borate and 2 mM EDTA) and visualized using a PHOSPHORIMAGER® (Molecular Dynamics, now GE Healthcare, Piscataway, N.J., U.S.A.). Markers were prepared as described previously (Sherman and Matson, 1994). Strand transfer reactions were performed in a manner similar to the oligonucleotide cleavage assay except after the 20-minute incubation, 1 μmol of a second unlabeled oligonucleotide of differing length containing the F plasmid nic site was added to the reaction and incubation was continued at 37° C. for 1 hour. The reaction was stopped and analyzed using the procedure described above. Liquid mating assays were performed as previously described (Matson et al., 2001) except HMS174 cells were utilized instead of HMS174 (DE3) to reduce the constitutive expression of the complementing protein. Briefly, cells containing pOX38TΔTraI and the appropriate complementing plasmid were grown overnight in the presence of appropriate antibiotics. Overnight cultures were used to inoculate cultures that were grown at 37° C. to mid-log phase (2-3 hours) in the absence of antibiotics. Donor cells were mixed (1:10) with recipient cells, incubated at 37° C. and then plated to select for transconjugants and counterselect for donors and recipients. Site-directed mutations in the traI gene were created using mutagenic primers and the site-directed mutagenesis protocol supplied by Stratagene, La Jolla Calif., U.S.A. pTYB2-traIN300 served as the template for PCR. The resulting clones were sequenced to confirm the presence of the engineered mutations and the absence of unintended mutations. A unique 700 bp NdeI-StuI fragment of traI containing the engineered mutations was removed from pTYB2-traIN300 and ligated into the full length traI gene in pET11c-traI that had been digested at unique NdeI and StuI sites to create the mutant pET11c-traI derivatives that were at utilized in genetic complementation assays.

Kinetic Assay Formulations

Reaction Buffer: 6.42 mM MgCl₂, 20.5% glycerol, 153.9 mM NaCl, and 51.3 mM Tris-HCl pH 7.5. Streptavidin Wash Buffer (Tris buffered saline, TBS/Tween): and 150 mM NaCl, 25 mM Tris-HCl pH 7.5, and 0.05% Tween-20. Stopping Buffer: 1.2% sodium-dodecyl sulfate (SDS), and 300 mM EDTA pH 10. TE Buffer: 1 mM EDTA, and 10 mM Tris-HCl pH 7.4. Fluorescence Buffer: 80% glycerol, 200 mM Tris-HCl pH 8.0. Short-term N300 stock (for storage at −20° C.): 50% Reaction Buffer, 49.8% glycerol, 0.2% long-term protein solution (84.6 nM final N300 concentration). All 5× stocks except Probe stocks were diluted in Reaction Buffer. 5× Enzyme Stock: 8.4 mL short-term N300 stock diluted to 2.02 nM N300. 5× Inhibitor Stocks: imidodiphosphate (pNp) at 0-50 nM in Reaction Buffer. 5× Substrate Stocks: oligonucleotides b29 and PCb31eco diluted to 19.6-158.5 mM each, by 3-fold serial dilutions. 5× Probe Stocks: 23.5-190.2 mM (1.2-fold molar excess) oligonucleotide downF in TE Buffer. All reactions and procedures involving downF were assembled or performed in darkened conditions. Solutions and microtiter plates containing downF were kept in foil-lined containers at all times to prevent photobleaching.

Fluorescence Kinetic Assays

Two oriT derived oligonucleotides, b29 and PCb31eco, were designed for binding and cleavage by TraI based on past studies (see Oligonucleotides section herein; FIG. 4A). The overall method is modified from those described previously for the study of TraI and R388TrwC (Byrd et al., 2002; Grandoso et al., 2000; Matson and Morton, 1991). Reactions were assembled from 16 μL of 5× Substrate Stock, 16 μL of 5× Inhibitor Stock, and 32 μL of Reaction Buffer. 80 μL reactions were initiated with 16 μL of 5× Enzyme Stock and raised to 37° C. 10 μL samples were removed at eight time points (optimized to define timecourses) and stopped in 10 μL of Stopping Buffer at room temperature. Stopped reactions were placed on a 100° C. heat block for one minute, spiked with 2 μL of 5× Probe Stock while still hot, and incubated at 37° C. for 10 minutes (see Kinetic Assay Formulations for probe handling).

Samples were diluted to 65 mL with Streptavidin Wash Buffer and transferred to Biobind Assembly streptavidin-coated microtiter plates (Thermo Electron Corporation, Waltham, Mass., U.S.A.) and incubated at 37° C. for 45 minutes. Plates were washed with at least 5-fold excess Streptavidin Wash Buffer (in an inverted position to prevent excess biotinylated species from transferring between wells). Wells were filled with 65 μL of Fluorescence Buffer for optimum 6-FAM™ fluorescence. Plates were read in a FLUOSTAR OPTIMA™ (BMG Labtech, Offenburg, Germany) with a 490 nm excitation and 520 nm emission filters (10 nm bandpass) with the gain optimized for maximum signal. The timecourses thus collected were designated Cleavage Courses. Plates were removed and irradiated with 306 nm ultraviolet radiation for 10 minutes to cleave the PC biotin linker. They were then washed inverted with at least 5-fold excess Streptavidin Wash Buffer, filled with 65 μL of Fluorescence Buffer, and again read as above. These timecourses were designated Cleavage+Crossover Courses (FIG. 4A).

Kinetic Data Processing

All timecourses were fitted by nonlinear regression with un-weighted least-squares methods using SIGMAPLOT™ 8.0 (SYSTAT Software, Inc., Point Richmond, Calif., U.S.A.) (also used for graph construction). Timecourses with blanked signals in arbitrary fluorescence units were scaled by curve mean in groups of at least four replicates, culled for outliers, and averaged by time point. On average, one replicate time point was culled from each group of four used. The averaged curves were fitted to a simple, three-parameter exponential decay equation (Equation 1), time versus signal, and converted into units of concentration.

signal=y₀+ae^{-bt} Equation 1

where t is time in seconds, y₀+a is the initial signal, b is a constant that determines the shape of the curve. Integration of the overall rate equation, which included several individual rate constants, for fitting of timecourses was prohibitive. The derivative of the decay equation at zero time was taken as the negative of the initial reaction velocity (v₀; equals ab from Equation 1). Michaelis/Menton curves (v₀ versus substrate concentration) were constructed for Cleavage and Cleavage+Crossover for each inhibitor concentration. Each Cleavage curve was subtracted from its corresponding Cleavage+Crossover curve to yield a Crossover curve (FIG. 4B).

Calculation of Kinetic Constants

Competitive (K_{ic}) and uncompetitive (Kᵢᵤ) inhibition constants were estimated from Vₘₐₓ^{app} and Kₘ^{app} values obtained with one of two methods: first, Cleavage and Crossover curves were fitted to the simple modern Michaelis/Menton equation (Equation 2) by nonlinear regression with un-weighted least-squares method in SIGMAPLOT® 8.0; second, the Cornish-Bowden/Eisenthal scale-free direct linear plot method was applied as implemented in the Exploratory Enzyme Kinetics Macro for SIGMAPLOT® 8.0 (Burnham and Anderson, 1998; Cornish-Bowden and Eisenthal, 1974; Cornish-Bowden and Eisenthal, 1978; Eisenthal and Cornish-Bowden, 1974; Willemoes et al., 2000).

$${v}_{0} = \frac{{V}_{max}^{app} S}{{K}_{m}^{app} + S} \qquad Equation 2$$

where Kₘ^{app} is the apparent Michaelis constant and Vₘₐₓ^{app} is the maximum reaction velocity. Competitive (K_{ic}) and uncompetitive (Kᵢᵤ) inhibition constants were estimated from extrapolation of the negative of ordinate intercepts of Kₘ^{app}/Vₘₐₓ^{app}-versus inhibitor concentration and 1/Vₘₐₓ^{app-}versus-inhibitor concentration, respectively. Definitions for Vₘₐₓ^{app} and Kₘ^{app} (Equations 4-5) were obtained by comparing the equation for mixed inhibition (Equation 3) with the Michaelis/Menton equation (Equation 1) and setting K_{ic} and Kᵢᵤ individually to infinity.

$$v = \frac{{V}_{max} S}{{K}_{m} ( 1 + I / {K}_{ic} ) + S ( 1 + I / {K}_{iu} )} \qquad Equation 3$$

where v is reaction velocity, S is substrate concentration, and I is inhibitor concentration.

$${V}_{max}^{app} = \frac{{V}_{max}}{1 + I / {K}_{iu}} , \\ {K}_{m}^{app} = \frac{{V}_{max} / {K}_{m}}{1 + I / {K}_{ic}} \qquad Equation 4 , 5$$

where Vₘₐₓ is the uninhibited maximum velocity and Kₘ is the uninhibited Michaelis constant (Table III). Note that kinetic assays for DNA cleavage and crossover by TraI N300 measure apparent reactions, given that “cleavage” and “crossover” are actually amalgams of all mechanistic paths leading to “cleaved” and “crossed” products. Thus, the Vₘₐₓ, Kₘ, and Kᵢ values reported are apparent constants.

TABLE III

| TraI N300 Kinetic Constants | | Vmax (nM/s) | Km (nM) | Kic (nM) | Kiu (nM) |
|---|---|---|---|---|---|
| Cleavage | M/M Nonlinear Regression | 0.555 ± 0.04 | 297 ± 3.12 | 2.3 ± 0.26 | 3.17 ± 0.32 |
| | C-B/E Direct Linear Plot | 0.647 ± 0.108 | 358 ± 29.91 | 2.42 | 2.65 |
| Crossover | M/M Nonlinear Regression | 0.152 ± 0.015 | 69.5 ± 26.7 | 0.885 ± 0.543 | −a |
| | C-B/E Direct linear Plot | 0.171 ± 0.096 | 75.2 ± 31.3 | 1.59 | 3.24 |

### Mating Assays

Mating assays were performed as described (Matson et al., 2001). Briefly, the donor strain JS10 contains pOX38T, a mini-F-plasmid capable of conjugative transfer, which also carries a Tet resistance gene, while the recipient strain JS4 is F− and Str^{r}. Donor and recipient strains from saturated overnight cultures grown under antibiotic selection were diluted 1:50 into LB and grown to an OD₆₀₀ of approximately 0.6 in the absence of selection at 37° C. Donors and recipients were then mixed at a ratio of one donor to nine recipients in the presence of the desired concentrations of pNp (e.g., 10, 100 μM, 1, 10 mM) and incubated at 37° C. for five minutes. Following this incubation, the mating mixtures were diluted 1:10 into LB containing the desired concentration of pNp and incubated for a further 30 minutes. Following mating, the mixtures were vortexed to disrupt mating pairs and serially diluted into 0.9% sterile saline. Dilutions, predicted to result in 300-500 colonies on positive control plates, were plated on LB-agar plates containing both streptomycin and tetracycline in order to select for transconjugants and counter select for donors and unmated recipients. Eight replicate samples of each final dilution were plated, the colony counts culled for outliers at 95% confidence, and then averaged.

### Cell Toxicity Assays

This assay is designed to expose the JS4 (F−), JS10 (F+), and JS11 (F+ but with traI deleted) strains to inhibitors under conditions which parallel the mating assay. Again, saturated overnight cultures grown under antibiotic selection were diluted 1:50 into LB and grown to an OD₆₀₀ of approximately 0.6 in the absence of selection at 37° C. Cultures were then incubated in the presence of the desired concentrations of pNp at 37° C. for 35 minutes, and then diluted into 0.9% sterile saline. Dilutions predicted to result in countable numbers of colonies were plated on LB-agar plates containing proper antibiotics.

Phylogenetic Analysis

Relaxase sequences were selected from non-redundant databases using PSI-Blast searches with default parameters (Altschul et al., 1997). The sequence containing the N-terminus to the last metal-chelating histidine of plasmid R64 NikB (NCBI accession BAA78021) was used for eight searching iterations, and 500 sequences with expectation values (e-values) of 7×10⁻¹⁰ were accepted. A second search began with the same sequence translated from Brevibacterium linens strain BL2 chromosomal locus BlinB01003615 (NCBI accession ZP_{—}00378014) and continued for three iterations before generating 500 additional sequences with e-values of 4×10⁻¹⁰ or better. NCBI GenPept descriptions were used to remove of redundant sequences and fragments. Sequences were named as follows: (protein name)_(plasmid or “Ch”+locus)_(genus-species code)_(strain), where the genus-species code is the first three letters of the genus and the first two of the species name (Tables IV and V). Annotated sequences were separated into 18 clusters in a ClustaIX alignment (Thompson et al., 1994) mimicking default Blast parameters, i.e. a BLOSUM matrix (Henikoff and Henikoff, 1992) with gap penalties 11/1 for opening/extension and no secondary structure masking (BLOSUM, 11/1, no ss). The cluster containing E. coli F plasmid TraI was aligned with secondary structure gap penalty masks derived from the low salt structure reported here (BLOSUM, 4/1, ss/TraI). Likewise, the cluster with R388 TrwC was aligned with a secondary structure mask assigned from PDB accession 1OMH (BLOSUM, 4/1, ss/TrwC). Remaining clusters were individually aligned with either BLOSUM (6/1, no ss) or Gonnet (10/0.2, no ss) matrices (Gonnet et al., 1992). Clusters were then aligned against one another in ClustalX profile alignments (Gonnet, 4/0.2, ss/TrwC), and all alignments were manually edited in BioEdit (Hall, 1999) to correct for gap border errors and misalignments (Tables IV and V). A maximum parsimony tree was constructed with the PHYLIP PROTPARS module (Felsenstein, 1989; Felsenstein, 1993) and displayed with TreeView (Page, 1996).

TABLE IV

| | | | Trial Residue Position | | | | | |
|---|---|---|---|---|---|---|---|---|
| 1 | Phylogeny | Tyrosine Constellation | 33′ | 52′ | 144′ | 81 | 146 | 277″ |
| Outgroups: IncP, I, X, Q-like Gram⁺ (65) IncQ (10) Legionella tra operons (4) Rhizobiales tra operons (17) pGOX1 TraA & other transitional forms (12) Actinobacteria plasmid tra operons (7) IncN, IncW, & IncP9 (12) IncF (12) | 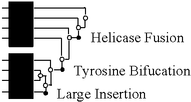 | Gp₂ApApxpYl GHSs₆Yp GpSs₅Yp (YN)cY(Y/L/R)x₂₀₋₃₀Y(Y/L)p VxYYx₁₂₋₂₄YYs Y(Y/F)X₂₋₄DpYYs YYx₃₋₂₄DNYYs | c R p W/Y W W W | −/x I n n V F/L F/L | A T/A V/A F/A Y F F/Y | −/p R R/S D/E D D D | H H H H/Q H H/Q H | — H H W W W W |

TABLE V

| | |
|---|---|
| gene locus species (modifier) | NCBI accession |

IncF

| | |
|---|---|
| tral F Escco | BAA97974 |
| tral p1658/97 Escco | AAO49548 |
| pre pE194 Escco | AAQ98619 |
| tral pAPEC-O2-R Escco | YP_190115 |
| tral R100 Escco | CAA39337 |
| tral pSLT Salty | AAL23509 |
| tral pG8786 Yerpe | YP_093987 |
| tral pED208 Salty | AAM90727 |
| tral pYJ016 Vibvu | NP_932226 |
| tral Ch VV20663 Vibvu | AAO07605 |
| tral pPBPR1 Phopr | CAG17960 |
| tral pLPP Legpn Paris | CAH17216 |

Actinobacteria plasmids

| | |
|---|---|
| traA pCE2 Coref | BAC19762 |
| traA pNG2 Cordi | NP_863184 |
| traA pTET3 Corgl | NP_478092 |
| traA pGA2 Corgl | AAO18209 |
| traA pCG4 Corgl | AAG00272 |
| traA pCE3 Coref | BAC19788 |
| tra pVT2 Mycav | AAL23621 |

Rhizobiales tra operons

| | |
|---|---|
| traA1 pSymA Sinme | NP_435751 |
| traA2 pSymB Sinme | CAC49066 |
| traA AT Agrtu | NP_535485 |
| traA p42d Rhiet | AAM54881 |
| traA Ch MBNC03003747 Messp | ZP_00193296 |
| traA pGOX3 Gluox | YP_190433 |
| traA Ch mll0964 Meslo | NP_102651 |
| traA Ch AE008200.1 Agrtu | AAK88593 |
| traA Ch Atu4855 Agrtu | NP_535333 |
| traA pRi1724 Agrrh | BAB16231 |
| traA pRiA4b Agrtu | BAB47249 |
| traA p42a Rhiet | AAO43541 |
| traA pNGR234a Rhisp | T02782 |
| traA Ti Agrtu | AAK91091 |
| traA pTiC58 Agrtu | AAC17212 |
| traA pTi-SAKURA Agrtu | BAA87734 |
| traA pHCGS Olica | CAG28509 |

IncN, P9, W

| | |
|---|---|
| trwC R388 Escco | CAA44853 |
| trwC pXcB Xanci | AAO72099 |
| trwC pXAC64 Xanax | NP_644759 |
| traC pWWO Psepu | NP_542915 |
| traC pDTG1 Psepu | NP_863125 |
| traC pBI709 Psepu | AAP57243 |
| tral pCU1 Escco | AAD27542 |
| tral R46 Salty | AAL13397 |
| trwC Ch ELI2871 Eryli | ZP_00377630 |
| hypo Ch ELI2182 Eryli | ZP_00376941 |
| tra Ch Saro02002574 Novar | ZP_00302710 |
| tral pLPL Legpn Lens | CAH17351 |

Transitional forms

| | |
|---|---|
| tra Ch Npun02004382 Nospu | ZP_00111530 |
| tral pCC7120gamma Nossp | NP_478459 |
| tra Ch Npun02003762 Nospu | ZP_00110457 |
| tral Ch lpg2077 Legpn Phil | YP_096090 |
| traA2 pAA1 Artau | AAS20144 |
| traA Ch Nocsp | AAV52093 |
| traA pKB1 Gorwe | NP_954808 |
| traA Ch BlinB01003615 Breli | ZP_00378014 |
| traA pGOX1 Gluox | YP_190362 |
| tra pFP11 Strsp | YP_220461 |
| tra pFP1 Strsp | YP_220493 |
| traA pNAC3 Biflo | NP_848156 |

Legionella tra operons

| | |
|---|---|
| traA Ch lpl0169 Legpn | CAH14398 |
| traA Ch lpp0183 Legpn | CAH11330 |
| traA Ch Legpn | AAG45149 |
| traA Ch lpg1241 Legpn | YP_095272 |

IncQ

| | |
|---|---|
| mobA pXF5823 Xylfa | AAK13432 |
| mobA DN1 Dicno | NP_073212 |
| mobA pIE1115 unceu | CAC05678 |
| mobA pIE1130 uncultured | CAB75594 |
| mobA pVM111 Pasmu | CAD55845 |
| mobA pFL190 Expve | AAV49024 |
| mobA RSF1010 Escco | CAA28520 |
| repB RSF1010 | AAB22064 |
| mobA pAB6 Neime | AAD31795 |
| mobA pSJ7.4 Neigo | AAO45530 |

Outgroups

| | |
|---|---|
| mobB pDOJH10L Biflo | AAN15156 |
| mobA pTB6 Biflo | BAD89595 |
| mobA pBLO1 Biflo | AAN31778 |
| mob pNAC2 Biflo | NP_848160 |
| mobB pKJ36 Biflo | AAG43281 |
| hypo pSF118-44 Lacsa | YP_163783 |
| traA pWCFS103 Lacpl | YP_133752 |
| hypo pSF118-20 Lacsa | YP_163738 |
| traA pMRC01 Lacla | AAC55993 |
| traA Ch Llacc01002488 Lacla | ZP_00381644 |
| traA pIP501 Plapl | AAA99466 |
| mobL Ch ECA1659 Erwca | YP_049758 |
| traA Ch plu1828 Pholu | NP_929105 |
| mobL Ch ECA2898 Erwca | YP_050989 |
| nes pSK41 Staau | AAC61938 |
| nes pGO1 Staau | AAB09712 |
| hypo Ch SC4349 Salen | YP_219336 |
| hypo Ch ECA1065 Erwca | YP_049172 |
| bmgA Ch BT3143 Bacth | AAO78249 |
| bmgA Ch BT1126 Bacth | AAO76233 |
| bmgA Ch BF3285 Bacfr | CAH08980 |
| bmgA Ch Bacfr | AAL29920 |
| mocA Tn4399 Bacfr | AAA98401 |
| hypo Ch PG0868 Porgi | AAQ66015 |
| bmgA Ch BT4622 Bacth | AAO79727 |
| hypo pBFY46 Bacfr | YP_087130 |
| mocA pBF9343 Bacfr | CAH05728 |
| orf1 Ch Bacth | AAG17843 |
| hypo Ch BF0132 Bacfr | YP_097415 |
| bmgA Ch BT0101 Bacth | AAO75208 |
| hypo Ch BF1249 Bacfr | CAH06968 |
| bmgA Ch BF1367 Bacfr | YP_098652 |
| bmgA Ch BT2305 Bacth | AAO77412 |
| hypo Ch PG1489 Porgi | AAQ66534 |
| hypo Ch BF1759 Bacfr | CAH07458 |
| pnf2870 pNF2 Nocfa | YP_122139 |
| virD2 pEN2701 Strsp | NP_862041 |
| mobAE Ch Lacla | Q48665 |
| mobAE1 ch LLMOBAMAT Lacla | CAA61995 |
| ltrB pRS01 Lacla | AAB06502 |
| pcfG pCF10 Entfa | YP_195793 |
| hypo pTEF2 Entfa | NP_817049 |
| mobA Ch gbs1121 Strag | NP_735567 |
| smc Ch Ssui801000289 Strsu | ZP_00333025 |
| virD2 Ch Ssui801000426 Strsu | ZP_00332895 |
| hypo Ch gbs 1338 Strag | NP_735775 |
| hypo Tn5252 Strag | NP_688252 |
| hypo Tn5252 Strpn | NP_345530 |
| rlx pIP834 Entfa | AAF72355 |
| rlx Ch EF2303 Entfa | NP_815959 |
| mobA/repB pRAS3.2 Aersa | AAK97758 |
| mobA/repB pRAS3.1 Aersa | AAK97751 |
| mobA pTF-FC2 Acife | A43256 |
| mobA pTC-F14 Acica | AAP04747 |
| nikB pCTX-M3 Citfr | NP_775011 |
| mobA pEL60 Erwam | AAQ97916 |
| nikB pO113 Escco | AAQ17653 |
| nikB pSERB1 Escco | AAT94234 |
| nikB R64 | BAA78021 |
| nikB Collb-P9 Escco | BAA75140 |
| nikB pSC138 Salen | AAS76381 |
| mobA pDC3000A Psesy | NP_808687 |
| mobA pRA2 Pseal | AAD40339 |
| mobA Ch PSPTO1093 Psesy | NP_790927 |
| tral R751 Entae | NP_044272 |

### Example 1

Characterization of Relaxase DNA Nucleotide Complexes The crystal structure of the N-terminal 300-amino acid region of the E. coli F plasmid relaxase TraI (N300) was determined and refined to 2.4 Å resolution (Table I). N300 harboring a Tyr-16-Phe mutation (N300-Y16F) was crystallized in the presence of a 9-mer sequence of ssDNA derived from the F plasmid oriT (5′-GGTGTAGGTG-3′ (SEQ ID NO: 1), with A representing the site of cleavage). Although the primary catalytic tyrosine was inactivated to phenylalanine (Y16F), the DNA oligonucleotide was cleaved by the enzyme during crystallization and only a single nucleotide, the −1 T (bold above), was observed in the active site. Weak electron density indicated that nucleotides upstream of the −1 T were still covalently linked to this nucleotide, but no density was observed for the downstream nucleotides. As discussed below, tyrosines other than Y16 in the TraI relaxase active site are capable of catalyzing transesterification reactions to cleave DNA oligonucleotides, which, without wishing to be bound by theory, is likely what happened in this case. The position of the bound nucleotide in these structures, however, revealed a key step in the reaction pathway and facilitated the development of a heretofore unknown comprehensive mechanism for this enzyme.

The TraI relaxase is composed of a central five-stranded antiparallel β-sheet core flanked by ten α-helices and an additional pair of two-stranded antiparallel β-sheets (FIG. 2A). The N300 TraI relaxase structures presented herein share 0.51 Å root-mean-square deviation (rmsd) over 248 equivalent Ca positions with the structure of the 36 kDa N330 region of TraI described previously (PDB 1P4D) (Datta et al., 2003). Both the N300 structures and the N330 structure contain a central disordered region composed of residues 236-266. In the structures of the IncW plasmid R388TrwC relaxase in complex with a ssDNA hairpin, this region folds into two α-helices that close over the DNA substrate (PDB 1OMH, 1 OSB, 1 QX0, and 1 S6M) (Guasch et al., 2003). The remainder of the TrwC relaxase is similar in structure to TraI N300, sharing 1.24 Å rmsd over 192 Cα positions. A more recent structure of TraI N330, with an ssDNA 22-mer lacking a hairpin, shows the 236-266 region ordered as in the TrwC structures, suggesting that this region orders to bind extended upstream DNA regardless of DNA secondary structure (Larkin et al., 2005).

The TraI active site contains four tyrosines (16, 17, 23, 24, with a Y16-to-F mutant in these structures), an aspartic acid (81), and three histidines (146, 157, and 159) that coordinate a bound metal ion (FIG. 2B). Of numerous ions and molecules tested, only a Mg²⁺ ion refined well in the 4-5 σ difference density observed in this site. No divalent metals were added to the protein buffers. Therefore, Mg²⁺, which is required for the transesterase activity of TraI (Byrd and Matson, 1997), and yields the highest level of nicking activity (Larkin et al., 2005), likely co-purified with the enzyme. The thymine base moiety of the −1 thymidine nucleotide is stacked on the Y16F side chain, and the 3′-hydroxyl group of the cleaved nucleotide occupies one of the octahedral coordination sites of the bound Mg²⁺ ion. Three other Mg²⁺ coordination sites are filled by H146, H157, and H159, and the fifth would be filled by the hydroxyl group on the Y16 side chain (were it not mutated to phenylalanine in the present study). Thus, only one magnesium coordination site is empty in these structures.

Superimposition of the TrwC and TraI active sites reveals significant differences in the positions of the tyrosine residues in the two structures (FIG. 2C). The first tyrosine of the TrwC quartet, Y18, occupies the position of the second TraI tyrosine (Y17); in this position both residues hydrogen bond with the side chain of Asp-81. The second TrwC residue (Y19) swings down to dock in a hydrophobic pocket formed in TraI by Trp-33, Phe-52 and Phe-144, residues that are conserved in the pump-type conjugative relaxases of known sequence. The third and fourth tyrosines of TrwC(Y26 and Y27) are either unobserved or exhibit high thermal displacement parameters (B factors), while the corresponding region and final two tyrosines in TraI (Y23 and Y24) are well structured and exhibit relatively low B factors. Similarly, the base moiety of the TrwC −1 thymidine (Thy25, the twenty-fifth base of the co-crystallized oligonucleotide) is located in a distinct position relative to that of the −1 T in TraI, although the 3′-hydroxyls of both bases are in close proximity (FIG. 2D). The TrwC tyrosine positions are related to those of TraI by one third of a turn about the axis of alpha helix A (αA; α1 in TrwC) (Guasch et al., 2003). Thus, the active sites of the pump-type relaxases are conformable in nature. These observations indicate that the TraI N300 relaxase structure presented herein represents the state of the enzyme just after the initial DNA cleavage event, while the TrwC structure represents the state of the enzyme prior to oriT ligation or crossover, as outlined in detail herein.

### Example 2

### Site-Directed Mutagenesis and Functional Assays

To further facilitate the development of a comprehensive conjugative relaxase mechanism, the roles played by TraI active site residues in enzyme function were examined by site-directed mutagenesis followed by DNA cleavage, religation and transfer assays (Table II). Mutation of Asp-81, which is completely conserved among identified pump-type relaxases and contacts one of the active site tyrosine in one structural snapshot, to asparagine reduces DNA strand cleavage, religation and transfer 2-, 10- and >33,000-fold, respectively (Table II). Truncating the equivalent residue in the TrwC relaxase (D85) to alanine was shown to reduce DNA transfer by 9.000-fold (Table II) (Guasch et al., 2003), a level similar to the asparagine mutation in TraI. Thus, the negative charge of this aspartic acid side chain is required for enzyme function. Lys-265, which was hypothesized to serve as a general base during the relaxase catalytic cycle, was mutated to methionine but generated no effect. The role of a Mg²⁺-chelating residue was then examined to determine whether the full 2+ charge on the metal ion is necessary for conjugation. While replacement of the Mg-chelating residue His-159 with glutamine (H159Q) lead to no change in enzyme function, replacement with glutamic acid (H159E) leads to a ten-fold drop in DNA cleavage, eliminates DNA religation (crossover), and reduces DNA transfer by 100,000-fold (Table II). Thus, the 2+ charge on the Mg ion is essential for enzyme activity. These data establish that a single negative charge at Asp-81 and a double positive charge at the magnesium are required for the DNA strand cleavage, religation and transfer functions of the F plasmid TraI relaxase.

TABLE II
Tral Relaxase Mutant Activity

| Mutant | Cleavage % | Crossover % | Transfer % |
|---|---|---|---|
| Wt | 100 | 100 | 100 |
| DB1N | 53 | 10 | 0.0030 |
| H159Q | 55 | 95 | nd |
| H159E | 8.5 | nd | 0.0010 |
| K265M | 100 | nd | 59 |

| Mutant | Transfer % | X-fold Transfer Decrease |
|---|---|---|
| Y16F | 0.12 | 850 |
| Y16V | 0.0035 | 29000 |
| Y17F | 20 | 4.9 |
| Y17V | 0.0030 | 34000 |
| Y23F | 68 | 1.5 |
| Y23L | 7.5 | 13 |
| Y24F | 100 | 1.0 |
| Y24V | 15 | 6.5 |

It has been reported that pump-type relaxases contain two catalytic tyrosines, equivalent to Tyr-16 and Tyr-23 in TraI. The presently disclosed subject matter extends this observation by examining the role of each of the four TraI active site tyrosines by replacing them with phenylalanine and leucine or valine, and determining the effects these alterations have on conjugative DNA transfer (Table II). The mutations of Tyr-16 to Phe and Val have the largest impact, resulting in 10³-fold and 10⁵-fold reductions in DNA transfer, respectively. These data indicate that both the tyrosine hydroxyl and the aromatic ring of this primary catalytic side chain are critical for relaxase function. The role of the aromatic ring is supported by the structure presented above, which revealed contacts between the conjugated rings of Tyr-16 and the thymine base in the oriT nicking site (see FIG. 2). The replacement of Tyr-17 with Val had a 10⁴-fold more significant impact on DNA transfer than Tyr-17's replacement with Phe. These results indicate that the aromatic ring on this side chain is also critical. Surprisingly, the mutations of Tyr-23 to Phe and to Leu had relatively little impact on DNA transfer, reducing it by only 1.5- and 10-fold, respectively. Thus, transfer does not critically depend on a second catalytic tyrosine, at least for the F plasmid. The mutation of the equivalent residue (Tyr-26) to Phe in the related TrwC relaxase of plasmid R388, however, reduced transfer by 10-fold (vs. 1.5-fold for TraI), suggesting that different plasmid systems may be more or less dependent on a second catalytic tyrosine. Finally, the replacement of Tyr-24 in F plasmid TraI with Phe had no impact on DNA transfer, while its replacement with Val resulted in a 10-fold reduction. Again, this indicates that the aromatic ring on Tyr-24 plays a role in enzyme function.

Taken together, these data reveal that the conjugated rings on the four TraI active site tyrosines are critical to the relaxase action of TraI. Further, these data suggest that two catalytic tyrosines may not be essential for the conjugative transfer of the F plasmid.

### Example 3

### Elucidation of Conjugative DNA Transfer Mechanism

A comprehensive 8-step mechanism for DNA transfer mediated by the pump relaxase TraI has been developed that accounts for available functional, mutagenesis, and structural data (FIG. 3). Directed by the other protein components of the relaxosome (Step 1), TraI, which contains both a relaxase and helicase, binds to the melted F plasmid oriT and positions the transfer (T) strand in the relaxase active site (Step 2). The first catalytic tyrosine of TraI (Y16) cleaves the T-strand and forms a 5′-phosphotyrosine intermediate and a free DNA 3′-hydroxyl (Step 3) (Byrd and Matson, 1997; Lanka and Wilkins, 1995; Matson et al., 1993; Pansegrau and Lanka, 1996; Sherman and Matson, 1994). The 5′→3′ helicase region of TraI then begins to travel along the nicked DNA strand to unwind the F plasmid (Reygers et al., 1991; Traxler and Minkley, 1988). This action allows a cellular replication complex to use the free 3′-hydroxyl to generate a new complementary strand (Step 4). Such rolling circle-type replication has been demonstrated directly for IncQ plasmid R1162 (Parker and Meyer, 2002). However, it is also possible that strand replacement is not directly linked to conjugative transfer; this situation is considered below. Regardless of the link between transfer and replication in the donor cell, the helicase region of TraI provides the required motive force to extrude the T-strand of the DNA through the conjugative septum and into the recipient cell (Matson et al, 2001). As the F plasmid is unwound by TraI, the plasmid rotates counterclockwise as indicated in FIG. 3. The first DNA sequence generated by oriT-initiated strand synthesis by the replisome adjacent to TraI is a newly formed, hybrid oriT (step 4).

After the F plasmid is fully unwound and the T-strand is extruded into the recipient cell, the hybrid oriT returns to TraI (Step 5). The second catalytic tyrosine (Y23) then cleaves the hybrid oriT and generates the second 5′-phosphotyrosine intermediate and a free 3′-hydroxyl (Gao et al., 1994), a functional step similar to the viral RCR Rep proteins (i.e., ΦX174 GpA; Hanai and Wang, 1993; van Mansfeld et al., 1979) (Step 6). It is important to note, as disclosed in detail herein, that two phosphate groups exist simultaneously in the active site at this step. This observation lead to the identification by applicants of the first relaxase inhibitor, as disclosed herein.

The 3′-hydroxyl generated by the second DNA cleavage reaction then attacks the first 5′-phosphotyrosine intermediate on Y16 to reseal the T-strand and release a closed-circular single-stranded copy of the plasmid into the recipient cell (Step 7) (Pansegrau and Lanka, 1996). Finally, the 3′-hydroxyl provided by the completion of DNA synthesis by the trailing replisome attacks the second 5′-phosphotyrosine intermediate to regenerate an intact duplex F plasmid within the donor cell, and the release TraI (Step 8). Thus, when replicative strand replacement is initiated from oriT and is concomitant with conjugation, two catalytic tyrosines are required to properly resolve DNA transfer.

It is not clear, however, that DNA replication in the donor cell is always directly linked to conjugative transfer. Indeed, it has been shown that conjugative DNA transfer is not obligatorily coupled to replacement strand synthesis in the donor (Kingsman and Willets, 1978). Replicative strand replacement can also be initiated at the site utilized for the replication of the plasmid during typical bacterial cell growth, the F plasmid origin of vegetative replication (oriV). If strand replacement is not concomitant with conjugation, the mechanism presented in FIG. 3 proceeds as drawn through Step 4, including the extrusion of the T-strand into the donor cell. After extrusion is complete, though, the relaxase would return not to a hybrid oriT but to an oriT half-site, as shown in Step 5′. The 3′-hydroxyl in this oriT half-site simply reverses the first 5′-phosphotyrosine intermediate on Tyr-16, and releases two ssDNA products: the T-strand in the recipient cell, and the template strand in the donor cell. Both products would then be converted to dsDNA by cellular replication processes. Note that only one catalytic tyrosine would be required for this mode of conjugative transfer, in which it is disconnected from oriT-initiated strand replacement synthesis.

The active site of TraI is proposed to perform these precise DNA cleavage and religation (crossover) events by adopting three distinct states. The first state mediates the initial DNA strand nicking reaction prior to plasmid unwinding by the helicase. In this state, Tyr-16 is positioned adjacent to the Mg²⁺ ion with Tyr-17 docked 8 Å away (Mg⁺² to Tyr hydroxyl) and hydrogen-bonded to D81, as observed in the structure of the TraI N330 fragment described previously (PDB 1P4D) (Datta et al., 2003), and with the scissile phosphate coordinated to the magnesium as seen more recently (Larkin et al., 2005) (Step 2). Just after the first cleavage event, the thymine base of the −1 nucleotide stacks upon Y16, as observed in the TraI structure described herein (Step 3; FIG. 2). The second state occurs after F plasmid unwinding by the helicase. The 5′-phosphotyrosine linkage on Tyr-16 shifts one third of a turn about alpha helix A (αA) to dock adjacent to Asp-81, as observed in TrwC structures (PDB 1OMH, 1OSB, 1QX0, and 1S6M) (Steps 5 and 5′; FIG. 2). Finally, in the third state, Tyr-23 swings over and generates the second 5′-phosphotyrosine intermediate (Step 6). The placement of Tyr-16 in this state between Asp-81 and the Mg²⁺ makes it a good leaving group because it is properly polarized for cleavage (Step 7).

Taken together, this comprehensive DNA transfer mechanism addresses both the detailed catalytic steps in the active site as well as the global process of DNA transfer by extrusion into the recipient cell. In addition, it makes two important determinations about the TraI relaxase: two phosphotyrosine intermediates can be accommodated simultaneously, and the active site tyrosines are repositioned during the catalytic cycle.

### Example 4

### In Vitro Analysis of Relaxase Inhibition

The prediction that the TraI active site is capable of accommodating two phosphotyrosine intermediates led applicants to hypothesize that a net negatively-charged compound, such as for example a compound having a net charge of −2, including a diphosphonate or diphosphate would inhibit DNA cleavage and religation by the relaxase enzyme. To test this, a fluorescence-based assay was developed by applicants to examine the kinetics of DNA cleavage and crossover catalyzed by TraI (FIG. 4A). To measure cleavage, the disappearance of fluorescence from a 6-FAM™ 5′-end-labeled oligonucleotide was monitored over time. Crossover, represented by Step 7 in FIG. 4, was monitored by the addition of a labeled fragment to a previously unlabelled photocleavable fragment over time (FIG. 4A). Four replicates of eight time points for five ssDNA substrate concentrations were collected for each experiment (uninhibited and two concentrations of candidate inhibitor). This fluorescence assay, performed in a 96-well microtiter plate, was developed to facilitate the collection of the kinetic time courses, which would otherwise not have been feasible with standard polyacrylamide gel electrophoresis methods.

Kinetic results were examined by non-linear regression of the simple Michaelis/Menten equation (M/M NLR) and by the Cornish-Bowden/Eisenthal scale-free Direct Linear Plot method (C-B/E DLP), which produced similar results (Table III). TraI N300 exhibited apparent maximum velocity values (Vₘₐₓ^{app}) of 0.55-0.65 and 0.15-0.17 nM/sec, and apparent Michaelis constants (Kₘ^{app) of} 300-350 and 70-75 nM for cleavage and crossover, respectively (Table III). These are the first kinetic constants reported for a conjugative relaxase enzyme. The lower Kₘ observed for DNA crossover suggests that it may not be necessary to reassemble the relaxosome for cleavage of the hybrid oriT and formation of the second 5′-phosphotyrosine intermediate (Steps 5-6, FIG. 3). The binding of the second ssDNA to the TraI active site in preparation for crossover appears in vitro to be more favorable than the association of the initial ssDNA substrate.

An exemplary inhibitor, imidodiphosphate (pNp; FIG. 4B), was found to have a significant impact on the activity of TraI. Imidodiphosphate is one of the simplest, non-hydrolysable bis-phosphonates. As shown in FIG. 4B, 10 nM pNp significantly reduced the DNA cleavage activity of the enzyme. pNp was found to inhibit DNA cleavage with roughly equivalent inhibition constants (Kᵢ values) of 2.5-3 nM for both competitive and uncompetitive modes (K_{ic} and Kᵢᵤ, respectively), indicating that the compound is a noncompetitive inhibitor (Table III). For DNA crossover, pNp exhibited a higher Kᵢᵤ (3.2 nM) than K_{ic} (0.9-1.6 nM), indicating that the compound is a mixed inhibitor for this reaction, with competitive inhibition dominant (Table III). Pyrophosphate, the simplest hydrolyzable diphosphate, had no effect on enzyme activity (data not shown); thus, compounds having two covalently linked phosphates appear capable of inhibiting TraI. Thus, pNp is the first inhibitor described for a conjugative relaxase.

### Example 5

### Characterization of Relaxase

### pNp Complexes

The crystal structure of TraI relaxase N300 Y16F-DNA nucleotide-pNp ternary complex was determined and refined to 3 Å resolution using crystals grown in the presence of DNA, as above, and soaked in 1 mM pNp (FIG. 4C; Table I). A significant electron density peak was observed at a novel position adjacent to the bound Mg²⁺ ion in the structure. Although several candidate molecules were tested, only a single imidophosphonate moiety refined well in this position. The imidophosphonate moiety occupies the sixth octahedral coordination site of the bound magnesium. Three Mg coordination sites are filled by H146, H157, and H159, the fourth by the 3′ hydroxyl of the −1 thymidine, and the fifth by Y16 (mutated to F in this complex; FIG. 4C). It is likely that only one imidophosphonate moiety is observed because the crystal soaking method used to generate this structure did not allow the second pNp phosphonate group to displace the bound DNA nucleotide.

The distance from the imidophosphonate moiety to magnesium is 3.7 Å, slightly longer than the 3.3 Å average phosphorous to magnesium distance observed in monodentate chelation (based on (Harding, 2004)). However, the phosphonate refines with monodentate chelation and an oxygen-to-Mg distance of 2.6 Å, well within the recently established range of 1.7-2.9 Å. The covalent 5′-phosphotyrosine linkage on Y16 is expected to remain localized to that region of Mg coordination. Thus, the pNp structure reveals the position of a second phosphate binding site relevant to the 5′-phosphotyrosine linkage of Y23.

Taken together, the pNp structural and in vitro functional data (Example 4) establish that two phosphate groups can bind at once to the TraI transesterase active site, a feature exploited by the presently disclosed subject matter to inhibit DNA cleavage and religation by the pump-type relaxase TraI.

### Example 6

### In Vivo Analysis of pNp Relaxase Inhibition

Applicants next examined whether the relaxase inhibitor pNp is capable of impacting conjugative DNA transfer in vivo. Three E. coli strains with distinct F plasmid states were employed: JS4 (F−), JS10 (F+/traI+), and JS11, which contains a variant F plasmid in which the gene for TraI has been deleted (F+/traI−). Cells that lack the F plasmid (JS4, F−) are resistant to pNp (FIG. 4D; maroon), while the same cells including the F plasmid (JS10, F+/traI+) are effectively killed by the compound (FIG. 4D; yellow). However, if TraI is removed from F+ cells (JS11, F+/traI−), they become resistant to pNp (FIG. 4D; orange). Finally, DNA conjugation from F+(JS10) cells to F− (JS4) cells is also effectively inhibited by pNp (green), with the compound exhibiting an EC50 value of ˜10 μM.

### Example 7

### In Vivo Analysis of Relaxase Inhibition Using a Fluorescence Assay

The relaxase inhibitors pNp, iminobis, etidronate, clodronate, 1,2-bis(dimethoxyphosphoryl)benzene, and methylenediphosphonate disclosed herein were tested to determine if they are capable of impacting conjugative DNA transfer in vivo and whether these effects provide bactericidal activity.

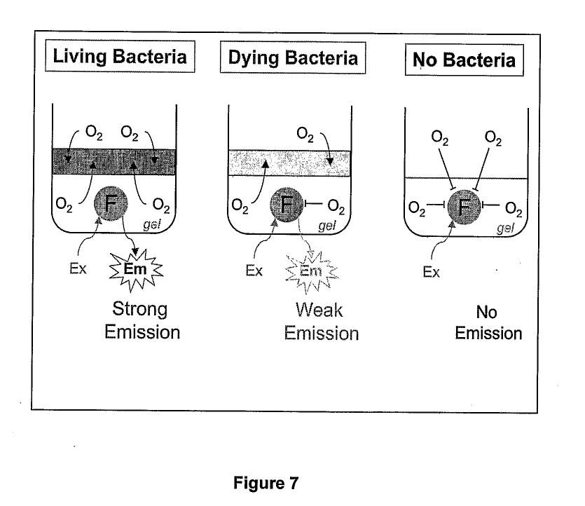

A 96-well fluorescence-based assay using oxygen biosensor plates (Becton Dickinson, Franklin Lakes, N.J., U.S.A.) was employed to monitor bacterial cell survival. FIG. 7 schematically illustrates the assay mechanism of action. Briefly, a tris 1,7-diphenyl-1,10 phenanthroline ruthenium (II) chloride fluorophore (Ex=485 nm, Em=510-630 nm) in a silicon-based hydrophobic gel exhibits fluorescence signal that correlates with the viability of the bacteria cultured in the well. In the absence of viable bacteria, however, no fluorescence emission is detected because the fluorophore is quenched by free oxygen. The assay can further be utilized to measure conjugative DNA transfer.

Relaxase-dependent cell survival was examined by placing cultures of F+/TraI+ E. coli cells (with antibiotic resistance, e.g., Ab^{r}₁, on a non-conjugative plasmid) within wells and exposing them to putative inhibitory compounds. As disclosed hereinabove, pNp not only inhibits conjugative DNA transfer in vivo but also selectively kills bacterial cells in a relaxase-dependent manner (see Example 6 and FIG. 4D). Negative controls for this assay can be F− (Ab^{r}₂) cells and F+ cells (Ab^{r}₁) containing either no F plasmid relaxase (TraI) gene, or a TraI harboring catalytic tyrosine to phenylalanine mutations; in both cases, these control cells should be resistant to relaxase-dependent cell toxicity. Positive controls can be of F+/TraI+ cells exposed to compounds previously determined to exhibit relaxase-dependent bactericidal activity, such as PNP. Fluorescence can be measured using a PHERASTAR™ fluorescence plate reader (BMG LABTECH, Offenburg, Germany) over 8 hours time.

Table VI shows data from experiments using the fluorescence assay to test pNp, iminobis, etidronate, clodronate, 1,2-bis(dimethoxyphosphoryl)benzene, and methylenediphosphonate for antimicrobial activity. Each of the tested compounds exhibited antimicrobial activity in a relaxase-dependent manner.

TABLE VI

| | | | | | | mass | | Selectivities | | | |
|---|---|---|---|---|---|---|---|---|---|---|---|
| Selectivities, Effective Concentrations, | | | | | | required | | by EC₅₀ | | | |
| and Minimum Inhibitory Concentrations | | 100 minute | | EC₅₀^{a} | | for 1.5 L^{b} | | Donors | Transconj. | 100 minute | |
| Iminobis(methylphosphonate) | vs. F⁺ donor cells | 10 | nM | 2 | ng/mL | 3 | μg | | 8.4E−03 | >10 | mM |
| PCNCP | vs. F⁻ recipient cells | 5 ± 3.3 | mM | 1 | mg/mL | 2 | g | 4.8E+05 | 4.0E+03 | >10 | mM |
| | vs. transconjugants | 1 | μM | 253 | ng/mL | 379 | μg | 1.2E+02 | | 8 ± 0.5 | mM |
| Etidronate | vs. F⁺ donor cells | 8 ± 0.2 | nM | 2 | ng/mL | 2 | μg | | 1.4E+00 | 20 | mM |
| ETIDRO | vs. F⁻ recipient cells | 327 | μM | 67 | μg/mL | 101 | mg | 4.3E+04 | 5.9E+04 | 42 | mM |
| | vs. transconjugants | 5 ± 1026.1 | nM | 1 | ng/mL | 2 | μg | 7.3E−01 | | 1 | mM |
| Clodronate | vs. F⁺ donor cells | 4 ± 91.2 | μM | 1 | μg/mL | 2 | mg | | 1.1E−01 | 30 | mM |
| CLODRO | vs. F⁻ recipient cells | 1 | mM | 365 | μg/mL | 548 | mg | 3.6E+02 | 3.9E+01 | >100 | mM |
| | vs. transconjugants | 38 | μM | 9 | μg/mL | 14 | mg | 9.2E+00 | | 5 | mM |
| 1,2-Bis(dimethoxy- | vs. F⁺ donor cells | 68 | nM | 20 | ng/mL | 30 | μg | | 4.8E−03 | >100 | mM |
| phosphoryl)benzene PBENP | vs. F⁻ recipient cells | 22 | mM | 7 | mg/mL | 10 | g | 3.3E+05 | 1.6E+03 | >100 | mM |
| | vs. transconjugants | 14 ± 0.3 | μM | 4 | μg/mL | 6 | mg | 2.1E+02 | | >100 | mM |
| Methylenediphosphonate | vs. F⁺ donor cells | 8 | μM | 1 | μg/mL | 2 | mg | | 8.8E+01 | 21 | mM |
| PCP | vs. F⁻ recipient cells | 667 ± 0.1 | μM | 117 | μg/mL | 176 | mg | 7.8E+01 | 6.9E+03 | 41 | mM |
| | vs. transconjugants | 96 ± 12.3 | nM | 17 | ng/mL | 25 | μg | 1.1E−02 | | 1 | mM |
| Imidodiphosphate | vs. F⁺ donor cells | 356 | nM | 63 | ng/mL | 94 | μg | | 6.7E−02 | 43 | mM |
| PNP | vs. F⁻ recipient cells | 593 ± 3.5 | μM | 105 | μg/mL | 157 | mg | 1.7E+03 | 1.1E+02 | 35 | mM |
| | vs. transconjugants | 5 | μM | 936 | ng/mL | 1 | mg | 1.5E+01 | | 9 | mM |

| | | | |
|---|---|---|---|
| | mass | Selectivities | |
| Selectivities, Effective Concentrations, | required | by EC₅₀ | 24 hr. MIC bounds^{c} |

| and Minimum Inhibitory Concentrations | | EC₅₀^{a} | | for 1.5 L^{b} | | Donors | Transconj. | lower | | upper | |
|---|---|---|---|---|---|---|---|---|---|---|---|
| Iminobis(methylphosphonate) | vs. F⁺ donor cells | >2 | mg/mL | >3 | g | | nd | 1.25 | mM | 10 | mM |
| PCNCP | vs. F⁻ recipient cells | >2 | mg/mL | >3 | g | nd | nd | | | | |
| | vs. transconjugants | 2 | mg/mL | 2 | g | nd | | | | | |
| Etidronate | vs. F⁺ donor cells | 4 | mg/mL | 6 | g | | 2.0E+01 | 1.56 | mM | 12.5 | mM |
| ETIDRO | vs. F⁻ recipient cells | 9 | mg/mL | 13 | g | 2.1E+00 | 4.1E+01 | | | | |
| | vs. transconjugants | 210 | μg/mL | 314 | mg | 5.1E−02 | | | | | |
| Clodronate | vs. F⁺ donor cells | 7 | mg/mL | 11 | g | | 6.1E+00 | 12.5 | mM | 100 | mM |
| CLODRO | vs. F⁻ recipient cells | >24 | mg/mL | >37 | g | nd | nd | | | | |
| | vs. transconjugants | 1 | mg/mL | 2 | g | 1.6E−01 | | | | | |
| 1,2-Bis(dimethoxy- | vs. F⁺ donor cells | >29 | mg/mL | >44 | g | | nd | 12.5 | mM | 100 | mM |
| phosphoryl)benzene PBENP | vs. F⁻ recipient cells | >29 | mg/mL | >44 | g | nd | nd | | | | |
| | vs. transconjugants | >29 | mg/mL | >44 | g | nd | | | | | |
| Methylenediphosphonate | vs. F⁺ donor cells | 4 | mg/mL | 6 | g | | 1.8E+01 | 1.56 | mM | 12.5 | mM |
| PCP | vs. F⁻ recipient cells | 7 | mg/mL | 11 | g | 1.9E+00 | 3.5E+01 | | | | |
| | vs. transconjugants | 205 | μg/mL | 307 | mg | 5.5E−02 | | | | | |
| Imidodiphosphate | vs. F⁺ donor cells | 8 | mg/mL | 11 | g | | 4.9E+00 | 12.5 | mM | 100 | mM |
| PNP | vs. F⁻ recipient cells | 6 | mg/mL | 9 | g | 8.0E−01 | 4.0E−00 | | | | |
| | vs. transconjugants | 2 | mg/mL | 2 | g | 2.0E−01 | | | | | |

^{a}= standard errors (SE) represent the upper error bound propogated from curves using the mean of three replicate cell counts (and standard deviation of the mean), the lower error bound being 10\hat{ }(2*log₁₀EC₅₀ − log₁₀SEᵤₚₚₑᵣ).
^{b}= ~volume of human gut.
^{c}= turbid (lower) or clear (upper) wells after 24 hr. incubation, 8x serial dilutions; bounds identical for F⁺ or F⁻ cells; same approx. concentration range as 100 min. EC₇₅s.

### REFERENCES

The references listed below as well as all references cited in the specification are incorporated herein by reference to the extent that they supplement, explain, provide a background for or teach methodology, techniques and/or compositions employed herein.
Abdel-Monem, M., Durwald, H., and Hoffmann-Berling, H. (1976). Eur J Biochem 65, 441-449.
Ahmed, A. M., Kawamoto, H., Inouye, K., Hashiwata, Y., Sakaki, M., Seno, M., and Shimamoto, T. (2005). J Med Microbiol 54, 867-872.
Altschul, S. F., Madden, T. L., Schaffer, A. A., Zhang, J., Zhang, Z., Miller, W., and Lipman, D. J. (1997). Nucleic Acids Res 25, 3389-3402.
Berkow et al., (1997) The Merck Manual of Medical Information, Home ed. Merck Research Laboratories, Whitehouse Station, N.J.
Brunger, A. (1998). Acta Crystallographica D 54, 905-921.
Burnham, K. P., and Anderson, D. R. (1998). Model Selection and Inference (New York, N.Y.: Springer-Verlag).
Byrd, D. R., and Matson, S. W. (1997). Mol Microbiol 25, 1011-1022.
Byrd, D. R., Sampson, J. K., Ragonese, H. M., and Matson, S. W. (2002). J Biol Chem 277, 42645-42653.
Campos-Olivas, R., Louis, J. M., Clerot, D., Gronenborn, B., and Gronenborn, A. M. (2002). Proc Natl Acad Sci USA 99, 10310-10315.
Collaborative Computing Project, N. (1994). Acta Crystallographica D 50, 760-763.
Cornish-Bowden, A., and Eisenthal, R. (1974). Biochem J 139, 721-730.
Cornish-Bowden, A., and Eisenthal, R. (1978). Biochim Biophys Acta 523, 268-272.
Dash, P. K., Traxler, B. A., Panicker, M. M., Hackney, D. D., and Minkley, E. G., Jr. (1992). Mol Microbiol 6, 1163-1172.
Datta, S., Larkin, C., and Schildbach, J. F. (2003). Structure (Camb) 11, 1369-1379.
DeLano, W. L. (2002). The PyMOL Molecular Graphics System.
Domart, A., Robineau, M., Stroh, A., Dubertret, L., Monsallier, J. F., and Modai, J. (1974). Ann Med Interne (Paris) 125, 915-918.
Duch et al., (1998) Toxicol. Lett. 100-101:255-263.
Dyda, F., and Hickman, A. B. (2003). Structure (Camb) 11, 1310-1311.
Ebadi, (1998) CRC Desk Reference of Clinical Pharmacology. CRC Press, Boca Raton, Fla.
Eisenthal, R., and Cornish-Bowden, A. (1974). Biochem J 139, 715-720.
Felsenstein, J. (1989). Cladistics 5, 164-166.
Felsenstein, J. (1993). PHYLIP (Phylogeny Inference Package) (Department of Genetics, University of Washington, Seattle, Wash.: Distributed by the author).
Firth, N., Ippen-Ihler, K., and Skurray, R. A. (1996). In Escherichia coli and Salmonella: cellular and molecular biology, F. C. Neidhardt, R. Curtiss, III, J. L. Ingraham, E. C. C. Lin, K. B. Low, B. Magasanik, W. S. Reznikoff, M. Riley, M. Schaechter, and H. E. Umbarger, eds. (Washington, D.C.: ASM Press), pp. 2377-2401.
Freireich et al., (1966) Cancer Chemother Rep. 50:219-244
Gao, Q., Luo, Y., and Deonier, R. C. (1994). Mol Microbiol 11, 449-458.
Gelvin, S. B. (2000). Annu Rev Plant Physiol Plant Mol Biol 51, 223-256.
Golubov, A., Neubauer, H., Nolting, C., Heesemann, J., and Rakin, A. (2004). Infect Immun 72, 5613-5621.
Gonnet, G. H., Cohen, M. A., and Benner, S. A. (1992). Science 256, 1443-1445.
Goodman et al., (1996) Goodman & Gilman's the Pharmacological Basis of Therapeutics, 9th ed. McGraw-Hill Health Professions Division, New York.
Grandoso, G., Avila, P., Cayon, A., Hernando, M. A., Llosa, M., and de la Cruz, F. (2000). J Mol Biol 295, 1163-1172.
Guasch, A., Lucas, M., Moncalian, G., Cabezas, M., Perez-Luque, R., Gomis-Ruth, F. X., de la Cruz, F., and Coll, M. (2003). Nat Struct Biol 10, 1002-1010.
Hall, T. A. (1999). Paper presented at: Nucl. Acids. Symp. Ser.
Hanai, R., and Wang, J. C. (1993). J Biol Chem 268, 23830-23836.
Harding, M. M. (2004). Acta Crystallogr D Biol Crystallogr 60, 849-859.
Henikoff, S., and Henikoff, J. G. (1992). Proc Natl Acad Sci USA 89, 10915-10919.
Hickman, A. B., Ronning, D. R., Kotin, R. M., and Dyda, F. (2002). Mol Cell 10, 327-337.
Ilyina, T. V., and Koonin, E. V. (1992). Nucleic Acids Res 20, 3279-3285. Jones, T., Zou, J., Cowan, S., and Kjeldgaard, M. (1991). Acta Crystallographica A 47, 110-119.
Katzung, (2001) Basic & Clinical Pharmacology, 8th ed. Lange Medical Books/McGraw-Hill Medical Pub. Division, New York.
Kingsman, A. and Willetts, N. (1978). J Mol Biol 122, 287-300.
Lahue, E. E., and Matson, S. W. (1988). J Biol Chem 263, 3208-3215.
Langer et al., Proc. Natl. Acad. Sci. USA 78:6633 (1981).
Lanka, E., and Wilkins, B. M. (1995). Annu Rev Biochem 64, 141-169.
Larkin C, Datta S, Harley M J, Anderson B J, Ebie A, Hargreaves V, Schildbach J F. (2005). Structure 13, 1533-1544.
Lederberg, J., and Tatum, E. L. (1946). Nature 153, 558.
Llosa, M., Gomis-Ruth, F. X., Coll, M., and de la Cruz Fd, F. (2002). Mol Microbiol 45, 1-8.
Matson, S. W., Nelson, W. C. and Morton, B. S. (1993). J. Bacteriol 175, 2599-2606.
Matson, S. W., and Morton, B. S. (1991). J Biol Chem 266, 16232-16237.
Matson, S. W., and Ragonese, H. M. (2005). J Bacteriol 187, 697-706.
Matson, S. W., Sampson, J. K., and Byrd, D. R. (2001). J Biol Chem 276, 2372-2379.
Oancea, C., Klare, I., Witte, W., and Werner, G. (2004). J Antimicrob Chemother 54, 232-235.
Page, R. D. M. (1996). Computer Applications in the Biosciences 12, 357-358.
Pansegrau, W., and Lanka, E. (1996). Prog Nucleic Acid Res Mol Biol 54, 197-251.
Parker, C., and Meyer, R. J. (2002). Mol Microbiol 46, 761-768.
PCT International Publication No. WO 93/25521.
Pingoud, A., Fuxreiter, M., Pingoud, V., and Wende, W. (2005). Cell Mol Life Sci 62, 685-707.
Pingoud, A., and Jeltsch, A. (2001). Nucleic Acids Res 29, 3705-3727.
Read, R. J. (1986). Acta Crystallographica Section A 42, 140-149.
Rees, C. E., and Wilkins, B. M. (1990). Mol Microbiol 4, 1199-1205.
Remington et al., (1975) Remington's Pharmaceutical Sciences, 15th ed. Mack Pub. Co., Easton, Pa.
Reygers, U., Wessel, R., Muller, H., and Hoffmann-Berling, H. (1991). Embo J 10, 2689-2694.
Sherman, J. A., and Matson, S. W. (1994). J Biol Chem 269, 26220-26226.
Speight et al., (1997) Avery's Drug Treatment: A Guide to the Properties, Choice, Therapeutic Use and Economic Value of Drugs in Disease Management, 4th ed. Adis International, Auckland/Philadelphia.
Sprague, G. F., Jr. (1991). Curr Opin Genet Dev 1, 530-533.
Thompson, J. D., Higgins, D. G., and Gibson, T. J. (1994). Nucleic Acids Res 22, 4673-4680.
Tosini, F., Visca, P., Luzzi, I., Dionisi, A. M., Pezzella, C., Petrucca, A., and Carattoli, A. (1998). Antimicrob Agents Chemother 42, 3053-3058.
Traxler, B. A., and Minkley, E. G., Jr. (1988). J Mol Biol 204, 205-209.
U.S. Pat. No. 5,326,902.
U.S. Pat. No. 5,234,933.
U.S. Pat. No. 6,180,082.
van Mansfeld, A. D., Langeveld, S. A., Weisbeek, P. J., Baas, P. D., van Arkel, G. A., and Jansz, H. S. (1979). Cold Spring Harb Symp Quant Biol 43 Pt 1, 331-334.
Waters, V. L., and Guiney, D. G. (1993). Mol Microbiol 9, 1123-1130.
Wei, Z. Q., Chen, Y. G., Yu, Y. S., Lu, W. X., and Li, L. J. (2005). J Med Microbiol 54, 885-888.
Willemoes, M., Hove-Jensen, B., and Larsen, S. (2000). J Biol Chem 275, 35408-35412.
Zechner, E. L., de la Cruz, F., Eisenbrandt, R., Grahn, A. M., Koraimann, G., Lanka, E., Muth, G., Pansegrau, W., Thomas, C. M., Wilkins, B. M., and et al. (2000). In The horizontal gene pool, C. M. Thomas, ed. (Amsterdam, The Netherlands: Harwood Academic Publishers), pp. 87-174.

It will be understood that various details of the invention may be changed without departing from the scope of the invention. Furthermore, the foregoing description is for the purpose of illustration only, and not for the purpose of limitation.

What is claimed is:

1. A method of treating a microbial infection in a subject, comprising administering to the subject an effective amount of a compound that modulates an enzymatic activity of a relaxase polypeptide.

2. The method of claim 1, wherein the microbial infection is a bacterial infection.

3. The method of claim 2, wherein the compound is a relaxase dependent antibiotic.

4. The method of claim 2, wherein the relaxase polypeptide is a Mob polypeptide.

5. The method of claim 4, wherein the relaxase polypeptide is a TraI polypeptide.

6. The method of claim 2, wherein the compound inhibits polynucleotide cleavage, polynucleotide religation, or both polynucleotide cleavage and polynucleotide religation enzymatic activities of the relaxase polypeptide.

7. The method of claim 1, wherein the microbial infection is a viral infection.

8. The method of claim 7, wherein the relaxase polypeptide is a Rep polypeptide.

9. The method of claim 7, wherein the compound inhibits replication of viral polynucleotide sequences.

10. The method of claim 1, wherein the compound is co-administered with at least one additional compound having antimicrobial activity.

11. The method of claim 1, wherein the compound has a net negative charge.

12. The method of claim 11, wherein the compound comprises a bis-phosphate, a bis-carboxylate, a bis-sulfate, or a bis-nitro moiety.

13. The method of claim 11, wherein the compound has the structure of Formula (I): 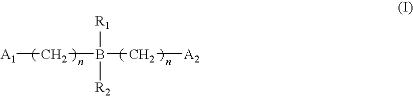
wherein:
n is an integer from 0 to 4;
A₁ and A₂ are independently selected from the group consisting of H, hydroxyl, alkyl, substituted alkyl, cycloalkyl, substituted cycloalkyl, aryl, substituted aryl, aralkyl, substituted aralkyl, phosphate, carboxylate, sulfate, and nitro, provided that at least one of A₁ or A₂ is phosphate, carboxylate, sulfate, or nitro;
B is selected from the group consisting of N, alkylene, substituted alkylene, cycloalkylene, substituted cycloalkylene, cycloalkenylene, substituted cycloalkenylene, arylene, and substituted arylene; and
R₁ and R₂ can each be present or absent and are independently selected from the group consisting of H, hydroxyl, halo, alkyl, substituted alkyl, cycloalkyl, substituted cycloalkyl, aryl, substituted aryl, aralkyl, and substituted aralkyl, or
a pharmaceutically acceptable salt thereof.

14. The method of claim 13, wherein the compound is selected from the group consisting of imidodiphosphate, methylenediphosphonate, etidronate, clodronate, pamidronate, alendronate, neridronate, iminobis, N-(2-hydroxyethyl)iminobis, glyphosine, 1,2-bis(dimethoxyphosphoryl)benzene, dichloromethylenediphosphonate, and SR12813.

15. The method of claim 1, wherein the subject is a mammal.

16. A method of inhibiting bacterial conjugation, comprising contacting a relaxase polypeptide within a bacterium with a relaxase dependent antibiotic, wherein the antibiotic modulates an enzymatic activity of the relaxase polypeptide.

17. The method of claim 16, wherein the relaxase polypeptide is a Mob polypeptide.

18. The method of claim 17, wherein the relaxase polypeptide is a TraI polypeptide.

19. The method of claim 16, wherein the antibiotic inhibits polynucleotide cleavage, polynucleotide religation, or both polynucleotide cleavage and polynucleotide religation enzymatic activities by the relaxase polypeptide.

20. The method of claim 16, wherein the antibiotic is co-administered to the bacterium with at least one additional antibiotic.

21. The method of claim 16, wherein the antibiotic has a net negative charge.

22. The method of claim 21, wherein the antibiotic comprises a bis-phosphate moiety, a bis-carboxylate moiety, a bis-sulfate moiety, or a bis-nitro moiety.

23. The method of claim 21, wherein the antibiotic has the structure of Formula (I): 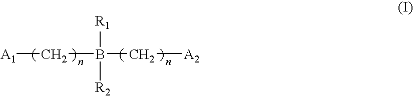
wherein:
n is an integer from 0 to 4;
A₁ and A₂ are independently selected from the group consisting of H, hydroxyl, alkyl, substituted alkyl, cycloalkyl, substituted cycloalkyl, aryl, substituted aryl, aralkyl, substituted aralkyl, phosphate, carboxylate, sulfate, and nitro, provided that at least one of A₁ or A₂ is phosphate, carboxylate, sulfate, or nitro;
B is selected from the group consisting of N, alkylene, substituted alkylene, cycloalkylene, substituted cycloalkylene, cycloalkenylene, substituted cycloalkenylene, arylene, and substituted arylene; and
R₁ and R₂ can each be present or absent and are independently selected from the group consisting of H, hydroxyl, halo, alkyl, substituted alkyl, cycloalkyl, substituted cycloalkyl, aryl, substituted aryl, aralkyl, and substituted aralkyl, or
a pharmaceutically acceptable salt thereof.

24. The method of claim 23, wherein the antibiotic is selected from the group consisting of imidodiphosphate, methylenediphosphonate, etidronate, clodronate, pamidronate, alendronate, neridronate, iminobis, N-(2-hydroxyethyl)iminobis, glyphosine, 1,2-bis(dimethoxyphosphoryl)benzene, dichloromethylenediphosphonate, and SR12813.

25. A compound of Formula (I): 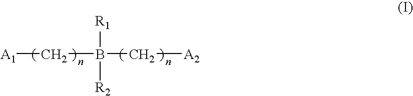
wherein:
n is an integer from 0 to 4;
A₁ and A₂ are independently selected from the group consisting of H, hydroxyl, alkyl, substituted alkyl, cycloalkyl, substituted cycloalkyl, aryl, substituted aryl, aralkyl, substituted aralkyl, phosphate, carboxylate, sulfate, and nitro, provided that at least one of A₁ or A₂ is phosphate, carboxylate, sulfate, or nitro;
B is selected from the group consisting of N, alkylene, substituted alkylene, cycloalkylene, substituted cycloalkylene, cycloalkenylene, substituted cycloalkenylene, arylene, and substituted arylene;
R₁ is selected from the group consisting of H, hydroxyl, halo, alkyl, substituted alkyl, cycloalkyl, substituted cycloalkyl, aryl, substituted aryl, aralkyl, and substituted aralkyl, and;
R₂ can each be present or absent and if present is selected from the group consisting of H, hydroxyl, halo, alkyl, substituted alkyl, cycloalkyl, substituted cycloalkyl, aryl, substituted aryl, aralkyl, and substituted aralkyl, or
a pharmaceutically acceptable salt thereof.

26. The compound of claim 25, wherein the compound has a structure selected from the group consisting of: 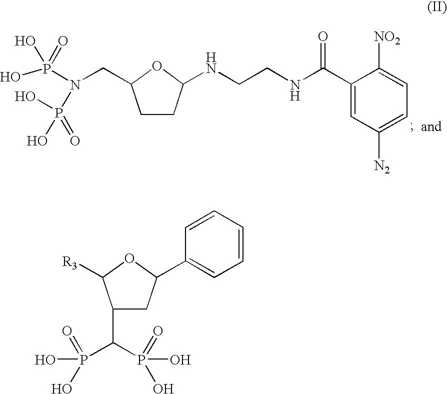
wherein:
R₃ is selected from the group consisting of H, hydroxyl, alkyl, substituted alkyl, cycloalkyl, substituted cycloalkyl, aryl, substituted aryl, aralkyl, and substituted aralkyl.

27. An assay method for measuring multiple catalytic kinetic time courses of a multifunctional polynucleotide-specific enzyme, comprising:
(a) providing:
i. a multifunctional polynucleotide-specific enzyme;
ii. a first substrate polynucleotide comprising a capture tag linked to a first end of the first polynucleotide, an enzyme recognition polynucleotide sequence, and a label linked to a second end of the first polynucleotide; and
iii. a second substrate polynucleotide comprising an enzyme recognition polynucleotide sequence and a cleavable capture tag linked to an end of the second polynucleotide;
(b) incubating the enzyme with the first polynucleotide and the second polynucleotide for a time sufficient to permit the enzyme to react with the first polynucleotide and the second polynucleotide;
(c) capturing the first polynucleotide and the second polynucleotide to a capture affinity molecule having binding affinity for both the capture tag and the cleavable capture tag, wherein the capture affinity molecule is bound to a substrate;
(d) washing the substrate to remove uncaptured molecules;
(e) determining a first kinetic time course of the enzyme based on a measured change in an amount of the label bound to the substrate over a time course;
(f) cleaving the cleavable capture tag, thereby releasing the second polynucleotide from the substrate; and
(g) determining a second kinetic time course of the enzyme based on a measured change in an amount of the label bound to the substrate before and after cleavage of the cleavable capture tag over a time course.

28. The method of claim 27, wherein the multifunctional polynucleotide-specific enzyme is a relaxase enzyme.

29. The method of claim 28, wherein the relaxase enzyme is a Mob relaxase enzyme.

30. The method of claim 29, wherein the capture tag is selected from the group consisting of biotin, digoxigenin, Protein A, Protein G, an oligonucleotide, a hapten, an antibody, and an anti-antibody-antibody.

31. The method of claim 27, wherein the cleavable capture tag is photocleavable biotin.

32. The method of claim 26, wherein the enzyme recognition polynucleotide sequence comprises a polynucleotide sequence homologous to a bacterial oriT polynucleotide sequence.

33. The method of claim 32, wherein the enzyme recognition polynucleotide sequence is a bacterial oriT polynucleotide sequence.

34. The method of claim 27, wherein the label is a fluorescent label.

35. The method of claim 27, wherein the label is bound to an end of a probe oligonucleotide having sequence homology to the second end of the first polynucleotide, wherein the probe oligonucleotide can hybridize to the first polynucleotide and thereby link the label to the first polynucleotide.

36. The method of claim 27, wherein the enzyme reacts with the first and second polynucleotides to cleave the polynucleotides, crossover ligate the polynucleotides, or both.

37. The method of claim 27, wherein the capture affinity molecule is selected from the group consisting of streptavidin, avidin, and an antibody.

38. The method of claim 27, wherein uncaptured molecules comprise fragments of the first and second polynucleotides cleaved by the enzyme from the first and second polynucleotides.

39. The method of claim 27, wherein determining the first and second kinetic time courses of the enzyme comprises determining the Vₘₐₓ, Kₘ, or both of the enzyme reactions with the first and second polynucleotides.

40. The method of claim 27, wherein the first kinetic time course is a measure of cleavage of the first polynucleotide by the enzyme.

41. The method of claim 27, wherein the second kinetic time course is a measure of crossover ligation of the first polynucleotide and the second polynucleotide by the enzyme.

42. A method of selecting for inhibitors of polynucleotide-specific enzymes, comprising:
(a) contacting a polynucleotide-specific enzyme with a substrate polynucleotide comprising a label in the presence of a candidate inhibitor;
(b) incubating the enzyme and the polynucleotide in the presence of the candidate inhibitor for a time sufficient to permit the enzyme to catalytically react with the polynucleotide;
(c) measuring a change in an amount of the labeled polynucleotide present over time, whereby the change in the amount of labeled polynucleotide correlates with an activity of the enzyme on the polynucleotide; and
(d) selecting the candidate inhibitor as an inhibitor of the enzyme if the activity of the enzyme on the polynucleotide is reduced in the presence of the candidate inhibitor as compared to a reaction in which the candidate inhibitor is absent.

43. The method of claim 42, wherein the polynucleotide-specific enzyme is a relaxase enzyme.

44. The method of claim 43, wherein the relaxase enzyme is a Mob relaxase enzyme.

45. The method of claim 44, wherein the substrate polynucleotide comprises a polynucleotide sequence homologous to a bacterial oriT polynucleotide sequence.

46. The method of claim 45, wherein the substrate polynucleotide is a bacterial oriT polynucleotide sequence.

47. The method of claim 42, wherein the candidate inhibitor has a net charge of −2.

48. The method of claim 42, wherein the candidate inhibitor comprises a bis-phosphate, a bis-carboxylate, a bis-sulfate, or a bis-nitro moiety.

49. The method of claim 42, wherein the label is a fluorescent label.

50. The method of claim 42, wherein the label is bound to an end of a probe oligonucleotide having sequence homology to the polynucleotide, wherein the probe oligonucleotide can hybridize to the polynucleotide and thereby link the label to the polynucleotide.

51. The method of claim 42, wherein the enzyme reacts with the polynucleotide to cleave the polynucleotide, ligate the polynucleotide, or both.

52. The method of claim 42, further comprising determining the Kᵢ, the mechanism of inhibition, or both, of the inhibitor on the enzyme.


SEQUENCE LISTING (4 sequences)

SEQ ID NO: 1 - DNA, 9 bp, Escherichia coli
ggtgtggtg

SEQ ID NO: 2 - DNA, 29 bp, Artificial sequence
  Artificially synthesized polynucleotide for kinetic assay, which can have a 5′ biotin label conjugated to the first nucleotide residue.
tttgcgtggggtgtggtgcttttgggtgg

SEQ ID NO: 3 - DNA, 31 bp, Artificial sequence
  Artificially synthesized polynucleotide for kinetic assays, which can have a 5′ photocleavable biotin conjugated to the first nucleotide residue.
ggaattctttttgcgtggggtgtgctgcttt

SEQ ID NO: 4 - DNA, 15 bp, Artificial sequence
  Artificially synthesized 15mer fluorescently labeled polynucleotide probe having  specificity for polynucleotides of the kinetic assay.
ccacccaaaagcacc